SEQUENCE LISTING (30 sequences)

SEQ ID NO: 1 - DNA, 4 bp, Artificial
  Synthetic construct
tata

SEQ ID NO: 2 - DNA, 8 bp, Artificial
  Synthetic construct
  misc_feature (1)..(4): n is a, c, g, t or u
nnnngatt

SEQ ID NO: 3 - DNA, 6 bp, Artificial
  Synthetic construct
  misc_feature (1)..(2): n is a, c, g, t or u
nnagaa

SEQ ID NO: 4 - DNA, 6 bp, Artificial
  Synthetic construct
  misc_feature (1)..(1): n is a, c, g, t or u
naaaac

SEQ ID NO: 5 - DNA, 38 bp, Artificial
  Synthetic construct
gctttatataaattagctgtaatggaggtattaaatat

SEQ ID NO: 6 - DNA, 38 bp, Artificial
  Synthetic construct
atatttaatacctccattacagctaatttatataaagc

SEQ ID NO: 7 - DNA, 55 bp, Artificial
  Synthetic construct
agcaaatgaaaaagattagctgtaagtttcagttgttagattatttggtatgtac

SEQ ID NO: 8 - DNA, 75 bp, Artificial
  Synthetic construct
gtaccaaataattaatgctctgtaatcatttaaaagtattttgaacggacctctgtttgacacgtctgaataact

SEQ ID NO: 9 - DNA, 48 bp, Artificial
  Synthetic construct
gctatttacttagtaattagctgtaatggagaaatccaaatgttaata

SEQ ID NO: 10 - DNA, 48 bp, Artificial
  Synthetic construct
tattaacatttggatttctccattacagctaattactaagtaaatagc

SEQ ID NO: 11 - DNA, 7 bp, Artificial
  Synthetic construct
aggaggu

SEQ ID NO: 12 - DNA, 7 bp, Artificial
  Synthetic construct
accaugg

SEQ ID NO: 13 - DNA, 7 bp, Artificial
  Synthetic construct
  misc_feature (5)..(5): n is a, c, g, t or u
  misc_feature (7)..(7): n is a, c, g, t or u
tatanan

SEQ ID NO: 14 - DNA, 6 bp, Artificial
  Synthetic construct
tataat

SEQ ID NO: 15 - DNA, 5 bp, Artificial
  Synthetic construct
ccaat

SEQ ID NO: 16 - DNA, 6 bp, Artificial
  Synthetic construct
  misc_feature (1)..(2): n is a, c, g, t or u
  misc_feature (4)..(5): n is a, c, g, t or u
nngnnt

SEQ ID NO: 17 - DNA, 7 bp, Artificial
  Synthetic construct
  misc_feature (1)..(4): n is a, c, g, t or u
nnnnaca

SEQ ID NO: 18 - DNA, 8 bp, Artificial
  Synthetic construct
  misc_feature (1)..(4): n is a, c, g, t or u
nnnngatt

SEQ ID NO: 19 - DNA, 4 bp, Artificial
  Synthetic construct
  misc_feature (4)..(4): n is a, c, g, t or u
tttn

SEQ ID NO: 20 - protein, 6 aa, Artificial
  Synthetic construct
GGGGGG

SEQ ID NO: 21 - protein, 5 aa, Artificial
  Synthetic construct
GGGGT

SEQ ID NO: 22 - protein, 6 aa, Artificial
  Synthetic construct
GGGPPP

SEQ ID NO: 23 - protein, 7 aa, Artificial
  Synthetic construct
GGGAPPP

SEQ ID NO: 24 - protein, 1629 aa, Artificial
  Synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 25 - protein, 20 aa, Artificial
  Synthetic construct
PIAIDLGVKNTGVFSAFYQK

SEQ ID NO: 26 - protein, 21 aa, Artificial
  Synthetic construct
MNNRTARRHQRRGIDRKQLVK

SEQ ID NO: 27 - protein, 34 aa, Artificial
  Synthetic construct
EWDKDTQQAISFLFNRRGFSFITDGYSPEYLNIV

SEQ ID NO: 28 - protein, 35 aa, Artificial
  Synthetic construct
KNIVDDNWQNIKQVLSAKHQLHIPIITESNAFEFE

SEQ ID NO: 29 - protein, 36 aa, Artificial
  Synthetic construct
LDHIIPRSHKKYGTLNDEANLICVTRGDNKNKGNRI

SEQ ID NO: 30 - protein, 31 aa, Artificial
  Synthetic construct
AKGDKPQASYSHLIDAMLAFCIAADEHRNDG

(19) United States
(12) Patent Application Publication
(10) Pub. No.: US 2021/0269801 A1
(43) Pub. Date: Sep. 2, 2021

(54) Methods of Managing Nucleic Acid Replication, Expression, and Cleavage Using CRISPR Associated Nucleases

(71) Applicant: Emory University, Atlanta, GA (US)

(72) Inventors: David Weiss, Decatur, GA (US); Hannah Ratner, Corvallis, OR (US)

(21) Appl. No.: 17/268,592

(22) PCT Filed: Aug. 14, 2019
(86) PCT No.: PCT/US2019/046585

§ 371 Date: Feb. 15, 2021

Related U.S. Application Data
(60) Provisional application No. 62/718,719, filed on Aug. 14, 2018.

(51) Int. Cl. C12N 15/113; C12N 9/22; C12N 15/74

(52) U.S. Cl. CPC C12N 15/113; C12N 2310/20; C12N 15/74; C12N 9/22

(57) ABSTRACT

This disclosure relates to methods of using a guide RNA sequence and CRISPR associated (Cas) nucleases for the purpose of managing replication of nucleic acids or expression of genes associated therewith. In certain embodiments, methods further optionally contemplate cleaving the nucleic acids at desired target sequences. Although it is not intended that certain embodiments of this disclosure be limited by any particular mechanism, is believed that shortening a guide sequence to partially hybridize with a target template strand prevents a guide RNA and Cas nuclease complex from catalyzing the cleavage of the nucleic acids and represses RNA transcription or protein expression.

### CROSS-REFERENCE TO RELATED APPLICATIONS

This application claims the benefit of U.S. Provisional Application No. 62/718,719 filed Aug. 14, 2018. The entirety of this application is hereby incorporated by reference for all purposes.

### STATEMENT REGARDING FEDERALLY SPONSORED RESEARCH OR DEVELOPMENT

This invention was made with government support under AI110701 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 18094PCT_ST25.txt. The text file is 22 KB, was created on Aug. 14, 2019, and is being submitted electronically via EFSWeb.

### BACKGROUND

CRISPR-Cas systems are native to bacteria and provide adaptive immunity against viruses and plasmids by cleaving associated nucleic acid sequences. Type-II CRISPR systems have a desirable characteristic in utilizing a single CRISPR associated (Cas) nuclease (specifically Cas9) in a complex with the appropriate guide RNAs (gRNAs). In bacteria, Cas9 guide RNAs comprise two separate RNA species: crRNA and tracrRNA. A target-specific CRISPR-activating RNA (crRNA) directs the Cas9/gRNA complex to bind and target a specific DNA sequence. The crRNA has two functional domains, a 5′-domain that is target specific and a 3′-domain that directs binding of the crRNA to the transactivating crRNA (tracrRNA). The tracrRNA is a longer, universal RNA that binds the crRNA and mediates binding of the gRNA complex to Cas9.

The gRNA function can be provided as an artificial single guide RNA (sgRNA), where the crRNA and tracrRNA are fused into a single species (see Jinek et al., Science 337, 816-21, 2012). The sgRNA format permits transcription of a functional gRNA from a single transcription unit that can be provided by a double-stranded DNA (dsDNA) cassette containing a transcription promoter. In mammalian systems, these RNAs have been introduced by transfection of DNA cassettes containing RNA Pol III promoters (such as U6 or H1) (see Xu et al., Appl Environ Microbiol, 2014. 80(5):1544-52).

Nishimasu et al., report a crystal structure of Cas9 in complex with sgRNA and its target DNA. Cell, 2014. 156(5):935-49. The crystal structure identified two lobes to the Cas9 enzyme: a recognition lobe (REC) and a nuclease lobe (NUC). The sgRNA:target DNA heteroduplex sits in a groove between the two lobes. The REC lobe interacts with the portions of the crRNA and tracrRNA that are complementary to each other.

Anders et al. (Nature, 2014, 513(7519) p. 569-73) elucidated the structural basis for DNA sequence recognition of protospacer associate motif (PAM) sequences by Cas9 in association with a sgRNA guide.

Price et al. (PNAS, 2015. 112 (19) 6164-6169) report Francisella novicida Cas9 (FnCas9) can be directed by an engineered RNA-targeting guide RNA to target and inhibit hepatitis C virus within eukaryotic cells.

Sampson et al. report a CRISPR/Cas system mediates bacterial innate immune evasion and virulence. Nature. 2013, 497(7448):254-7.

Zetsche et al. report Cpf1 is a single RNA-guided endonuclease of a class 2 CRISPR-Cas system. Cell. 2015, 163(3):759-71.

U.S. Patent Application Publication 20170247671 report Cas:guide RNA complexes for blocking off-target nucleic acids from cleavage by Cas:guide RNA complexes.

U.S. Patent Application Publication 20140295557 reports using truncated guide RNAs (tru-gRNAs) to increase specificity for RNA-guided genome editing. See also WO2017023974, WO2017015015, and WO2017180915.

References cited herein are not an admission of prior art.

### SUMMARY

This disclosure relates to methods of using a guide RNA sequence and CRISPR associated (Cas) nucleases for the purpose of managing replication of nucleic acids or expression of genes associated therewith. In certain embodiments, methods further optionally contemplate cleaving the nucleic acids at desired target sequences. Although it is not intended that certain embodiments of this disclosure be limited by any particular mechanism, is believed that shortening a guide sequence to partially hybridize with a target template strand prevents a guide RNA and Cas nuclease complex from catalyzing the cleavage of the nucleic acids and represses RNA transcription or protein expression.

In certain embodiments, this disclosure relates to methods of utilizing CRISPR-Cas systems to fine tune nucleic acids levels and sequences within the cell by: a) providing a Cas nuclease with a target double stranded nucleic acid sequence comprising a target segment adjacent to a protospacer adjacent motif (PAM) sequence or adjacent to a sequence with the reverse complement of the PAM; b) inserting into the cell a vector encoding an RNA sequence comprising a guide sequence under conditions such that the RNA sequence is expressed, or a vector containing a CRISPR array of multiple guiding spacers that can either be processed by the Cas nuclease, the existing cellular machinery, or non-native processing machinery delivered to the cell/system; wherein the guide sequence is identical to the target segment for as many nucleotides as are required to facilitate binding to the target, and wherein a Cas nuclease inside the cells in combination with the guide sequence represses replication of the nucleic acid without cutting either strand of the double stranded nucleic acid.

In certain embodiments, the guide sequence is identical to the target segment adjacent to a PAM, such that recognition of the PAM facilitates nuclease binding and guide RNA—target interaction. In certain embodiments, the target segment does not encode a protein. In certain embodiments, the target segment is in a 5′ untranslated region of a gene upstream of a start codon or in the promoter region of a gene. In certain embodiments, the PAM or reverse complement of the PAM is less than 100 nucleotides upstream from a start codon.

In certain embodiments, this disclosure contemplates using methods disclosed herein for transcriptional control using a guide RNA with a shortened target sequence in combination with a catalytically active Cas nuclease for altered repression and/or activation. For CRISPR-based repression, Cas nucleases are able to efficiently block transcriptional elongation or initiation in prokaryotes. Nuclease active Cas nucleases and guide RNA can be modified to improve interaction with a eukaryotic promoter and transcription initiation regions of the DNA. These modifications include fusing the catalytically active Cas nuclease to a Krüppel-associated box (KRAB), Max-interacting protein 1 (Mxi1), or four concatenated mSin3 interaction domains (SID4X). Transcriptional activation can be achieved by using the nuclease to prevent binding of a transcriptional repressor, or using the Cas protein to recruit transcriptional activators. Cas nucleases can be fused to various proteins to improve the efficiency of activation in different cell types and to recruit additional cellular proteins to specific sites on the DNA.

In certain embodiments, this disclosure contemplates using same Cas nuclease to both cleave DNA and control transcription, guided by RNAs of different lengths.

In certain embodiments, this disclosure contemplates using methods disclosed herein for multiplexing. In certain embodiments, this disclosure contemplates expressing a Cas nuclease with multiple different guides multiplexed for different targets. In certain embodiments, this disclosure contemplates using a catalytically active Cas nuclease with different types of guide RNAs, wherein the Cas nuclease could be multiplexed for many different functions that that could be executed simultaneously by a single type of Cas nuclease include spacers designed to cleave, repress, activate, detect, and degrade single or double stranded nucleic acids, e.g. dsDNA and ssDNA.

In certain embodiments, controlling, modulating, replication of a double stranded nucleic acid is repressing RNA transcription or gene expression, or activating RNA transcription or gene expression.

In certain embodiments, the PAM adjacent sequence on the target DNA is identical to the guide RNA for 5 or more nucleotides, but with less than 90% identity between the guide RNA and a 19 or more nucleotide region of the target, or partial or perfect identity between the guide RNA and less than 15, 16, 17 or 18 nucleotides of the target.

In certain embodiments, this disclosure relates to in vitro or in vivo methods of repressing replication of a double stranded nucleic acid, e.g., repressing ssRNA transcription from dsDNA, without cleaving or nicking the nucleic acid.

In certain embodiments, methods disclosed herein can be used with modified Cas nucleases that enable or enhance recruitment of transcription factors in eukaryotic cells or other cell types, such that the Cas nuclease can activate or repress, or generally fine tune RNA levels. In certain embodiments, the disclosure contemplates using a modified Cas nuclease that is either codon optimized for its target cell type, modified with epitope tag or NLS, or contains a mutation that alters function without preventing guide RNA binding.

In certain embodiments, this disclosure relates to in vitro or in vivo methods of repressing replication of a double stranded nucleic acid comprising: a) providing a cell with a double stranded nucleic acid sequence comprising a target segment adjacent to a protospacer adjacent motif (PAM) sequence or adjacent to a sequence with the reverse complement of the PAM; b) inserting into the cell an RNA sequence comprising a guide sequence or a vector encoding an RNA sequence comprising a guide sequence under conditions such that the guide RNA sequence is expressed, wherein the guide sequence is identical to the target segment for 6 or more nucleotides but less than 17 nucleotides in length, and wherein a Cas nuclease, capable of nicking or cleaving double stranded nucleic acids, inside the cell in combination with the guide RNA represses replication of the nucleic acid without cutting either strand of the double stranded nucleic acid.

In certain embodiments, the guide sequence has a 5-6 nucleotide seed sequence adjacent to the PAM that is identical and consecutive, but after that there can be 3 or more mismatches over a 20 nucleotide sequence total (including the seed sequence).

In certain embodiments, this disclosure relates to in vitro or in vivo methods of repressing replication of a double stranded nucleic acid comprising: a) providing a cell with a double stranded nucleic acid sequence comprising target segment adjacent to a protospacer adjacent motif (PAM) sequence or adjacent to a sequence with the reverse complement of the PAM; b) inserting into the cell a vector encoding an RNA sequence comprising a guide sequence linked to tracrRNA under conditions such the RNA sequence is expressed in the cell, wherein the guide sequence is identical to the target sequence for 6 or more nucleotides but less than 17 nucleotides in length, wherein a Cas nuclease inside the cells represses replication of the nucleic acid without cutting either strand of the double stranded nucleic acid.

In certain embodiments, this disclosure relates to in vitro or in vivo methods of repressing replication of a double stranded nucleic acid comprising: a) providing a cell with a double stranded nucleic acid sequence comprising target segment adjacent to a protospacer adjacent motif (PAM) sequence or adjacent to a sequence with the reverse complement of the PAM; b) inserting into the cell a vector encoding an RNA sequence comprising a guide sequence and tracrRNA under conditions such the guide sequence and tracrRNA is expressed in the cell, wherein the guide sequence is identical to the target sequence for 6 or more nucleotides but less than 17 nucleotides in length, wherein a Cas nuclease inside the cells represses replication of the nucleic acid without cutting either strand of the double stranded nucleic acid.

In certain embodiments, the RNA bases that match a target segment are contiguous. In certain embodiments, the RNA bases that match a target segment do not necessarily need to be in a row, and optionally the nucleotide at least contain 6 or more nucleotides but less than 17 nucleotides in length that match by identity, hybridization, base pairing over the span of the target.

In certain embodiments, homology between the target and the guide does not need to be consecutive, as long as there is a 5 nucleotide seed sequence that is identical or identical hybridization match to the guide RNA/adjacent to the PAM, bases that are not identical or identical hybridization match to the target can be interspaced at different locations in the rest of the guide sequence, or the guide can be truncated to less than 17, 18, or 19 bases but identical or identical hybridization match to the target, or truncated with other mutations.

In certain embodiments, the RNA bases that match may seed from five, six, or seven contiguous bases in a row directly adjacent in 5′ or 3′ of the PAM or the reverse complement of the PAM in order to initiate binding.

In certain embodiments, the PAM on either a sense strand and/or a template strand can be targeted to repress transcription.

In certain embodiments, the PAM is sufficient to facilitate Cas nuclease interaction with its target and is inclusive for nucleases that are engineered to recognize different PAMs as certain Cas nucleases recognize a range of PAM sequences with different affinities.

In certain embodiments, the guide RNA sequence is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon. In certain embodiments, the Cas nuclease is Cas9 or Cpf1. In certain embodiments, the method further comprises inserting into the cell a Cas nuclease or a vector encoding the Cas nuclease in operable combination with a promoter.

In certain embodiments, repressing replication of a double stranded nucleic acid is repressing RNA transcription or gene expression.

In certain embodiments, inserting into the cell an RNA sequence is inserting a vector encoding the RNA sequence.

In certain embodiments, this disclosure relates to methods of cleaving a double stranded nucleic acid comprising the steps disclosed herein for repressing replication without cleavage of a target sequence further comprising inserting into the cell a second RNA sequence, or vector encoding the second RNA sequence, comprising a guide sequence under conditions such that the second RNA is expressed and wherein a Cas nuclease is inside the cell cleaves one or both strands of the double stranded nucleic acid of the target sequence.

In certain embodiments, this disclosure relates to methods of repressing replication of a double stranded nucleic acid comprising: a) providing a cell with a double stranded nucleic acid sequence comprising target segment adjacent to a protospacer adjacent motif (PAM) sequence or adjacent to a sequence with the reverse complement of the PAM; b) inserting into the cell an RNA sequence comprising a guide sequence linked to tracrRNA, wherein the guide sequence is identical to the target sequence for 6 or more nucleotides but less than 17 nucleotides in length, wherein a Cas nuclease inside the cell capable of nicking or cleaving double stranded nucleic acids, in an area of hybridization with the guide sequence, represses replication of the nucleic acid without cutting either strand of the double stranded nucleic acid.

In certain embodiments, this disclosure relates to methods of repressing replication of a double stranded nucleic acid comprising: a) providing a cell with a double stranded nucleic acid sequence comprising a target segment adjacent to a protospacer adjacent motif (PAM) sequence or adjacent to a sequence with the reverse complement of the PAM; b) inserting into the cell a vector encoding an RNA sequence comprising a guide RNA sequence, wherein the guide RNA is identical to the target segment for 6 or more nucleotides but less than 17 nucleotides in length, and wherein a Cas nuclease inside the cells in combination with the guide RNA represses replication of the nucleic acid without cutting either strand of the double stranded nucleic acid.

In certain embodiments, the guide RNA is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon. In certain embodiments, the Cas nuclease is Cas9 or Cpf1. In certain embodiments, the method further comprising inserting into the cell a Cas nuclease or a vector encoding the Cas nuclease in operable combination with a promoter.

In certain embodiments, this disclosure relates to methods of nicking or cleaving a double stranded nucleic acid comprising the steps provided herein for repressing replication, further comprising inserting into the cell a second vector encoding a second RNA sequence comprising a guide RNA under conditions such that the second RNA is expressed and wherein a Cas nuclease inside the cells cleaves one or both strands of the double stranded nucleic acid.

In certain embodiments, methods of repressing replication of a double stranded nucleic acid comprises: a) providing a cell with a double stranded nucleic acid sequence comprising a target segment adjacent to a protospacer adjacent motif (PAM) sequence or adjacent to a sequence with the reverse complement of the PAM; b) inserting into the cell a vector encoding an RNA sequence comprising a guide sequence and a segment capable of hybridizing to a tracrRNA under conditions such that the expression of the RNA sequence forms a complex with a tracrRNA in the cell providing a guide RNA, wherein the guide RNA is identical to the target segment for 6 or more nucleotides but less than 17 nucleotides in length, and wherein a Cas nuclease inside the cells represses replication of the nucleic acid without cutting either strand of the double stranded nucleic acid.

In certain embodiments, there can be complementarity over a span of 20 nucleotides, as long as it is not perfect complementarity. In certain embodiments complementary is at most of 15 or 16 out of 20, with the requirement of 4, 5, or 6 base pairs in the seed are a perfect match.

In certain embodiments, the guide RNA is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon.

In certain embodiments, Cas nuclease is Cas9 or Cpf1. In certain embodiments, the PAM sequence is selected from Streptococcus pyogenes, e.g., NG or NGG, NNNNGATT (SEQ ID NO: 2), Streptococcus thermophiles, e.g., NNAGAA (SEQ ID NO: 3), Treponema denticola, e.g., NAAAAC (SEQ ID NO: 4), Staphylococcus aureus, e.g., NNGRRT (SEQ ID NO: 16), Campylobacter jejuni, e.g., NNNNACA (SEQ ID NO: 17), Neisseria meningitides, e.g., NNNNGATT (SEQ ID NO: 18), and Francisella orthologs, e.g., TNN or TTTN (SEQ ID NO: 19). In certain embodiments, the Cas nuclease is Cpf1 and the PAM is Streptococcus pyogenes NG or Francisella orthologs TTN. Other contemplated orthologs of Cpf1 are lactobacillus and acidaminococcus.

In certain embodiments, guide RNAs can be encoded within the same CRISPR array, or they can be encoded on separate vectors, or on the same vector under different promoters, depending on the use. For example, Cpf1 can process its own CRISPR array into individual guide RNAs

In certain embodiments, Cas nucleases such as Cas9 orthologs can degrade ssDNA or ssRNA. Thus, in certain embodiments, this disclosure contemplates multiplexing the cleavage and repression functions with guide RNAs that also guide these functions. In certain embodiments, this disclosure contemplates using these Cas nucleases to bind but not cleave with a partial RNA can also be utilized to detect nucleic acids (another function the same Cas nuclease can be used for based on its RNA guide, and thus can be multiplexed for) e.g., dCas9 or active Cas9 with a partial guide RNA.

In certain embodiments, methods disclosed herein further comprise inserting into the cell a Cas nuclease or a vector encoding the Cas nuclease in operable combination with a promoter.

In certain embodiments, methods disclosed herein further comprise inserting into the cell a single guide RNA or a vector encoding the single guide RNA in operable combination with a promoter.

In certain embodiments, methods disclosed herein further comprise inserting into the cell a tracrRNA or a vector encoding the tracrRNA in operable combination with a promoter.

In certain embodiments, methods disclosed herein further comprise inserting into the cell a guide RNA or a vector encoding the guide RNA in operable combination with a promoter wherein the guide RNA comprises a guide sequence linked to a crRNA and optionally tracrRNA.

In certain embodiments, methods disclosed herein further comprise inserting into the cell a guide RNA or a vector encoding the guide RNA in operable combination with a promoter wherein the guide RNA comprises a guide sequence linked to a segment of RNA that is capable of specific binding with a Cas nuclease such as a Cas9 or a Cpf1.

In certain embodiments, repressing replication of a double stranded nucleic acid is repressing RNA transcription or gene expression. In certain embodiments, the RNA is mRNA or microRNA. In certain embodiments, the double stranded nucleic acid is DNA or RNA.

In certain embodiments, this disclosure contemplates two vectors, one encoding for the purpose of nucleic acid cleavage and a second for the purpose of nucleic acid transcriptional repression.

In certain embodiments, a method of nicking or cleaving a double stranded nucleic acid comprises the steps disclosed herein or repressing replication of a double stranded nucleic acid, further comprising inserting into the cell a second vector encoding an RNA sequence comprising a guide RNA or guide sequence and a segment capable of hybridizing to a tracrRNA under conditions such that the guide RNA sequence is expressed and wherein a Cas nuclease inside the cells cleaves one or both strands of the double stranded nucleic acid.

In certain embodiments, a method of nicking or cleaving a nucleic a double stranded nucleic acid comprises the steps disclosed herein, further comprising inserting into the cell a second vector encoding an RNA sequence comprising a guide sequence linked to tracrRNA under conditions such that the RNA sequence is expressed in the cell and wherein a Cas nuclease inside the cells cleaves at least one stranded of the double stranded nucleic acid.

In certain embodiments, this disclosure relates to methods of nicking or cleaving a double stranded nucleic acid comprising the steps disclosed herein for repressing replication of a double stranded nucleic acid and further comprising: a) providing a cell with a double stranded nucleic acid sequence comprising target segment adjacent to a protospacer adjacent motif (PAM) sequence or adjacent to a sequence with the reverse complement of the PAM; b) inserting into the cell a vector encoding an guide RNA sequence under conditions such the RNA sequence is expressed in the cell, wherein the guide sequence is identical to the target sequence for more than 17 nucleotides in length, and wherein a Cas nuclease inside the cell cleaves at least one stranded of the double stranded nucleic acid.

In certain embodiments, the guide sequence is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon.

In certain embodiments, the sense strand or template strand target sequence does not encode a protein. In certain embodiments, the target sequence contains the start codon of a protein. In certain embodiments, the target sequence is an untranslated region up-stream from a start codon, such as in a promotor region. In certain embodiments, the target sequence comprises sequences of cis-acting regulatory elements such as promotor, enhancer, and silencer sequences. In certain embodiments, the target sequence comprises a ribosome binding site (RBS) sequence, Shine Dalgarno sequence AGGAGGU (SEQ ID NO: 11), Kozak consensus sequence ACCAUGG (SEQ ID NO: 12), or contains the start codon. In certain embodiments, the target sequence comprises a TATA (SEQ ID NO: 1) box, TATAWAW (SEQ ID NO: 13), where W is either A or T, Pribnow box, TATAAT (SEQ ID NO: 14), CCAAT (SEQ ID NO: 15) box.

With regard to any of the embodiments disclosed herein, the target segment is in a 5′ untranslated region of a gene upstream of a start codon or transcription start site such as a promotor region sequence. In certain embodiments, the target segment has a PAM or reverse complement of the PAM is less than 20 nucleotides upstream from a start codon. In certain embodiments, the target segment is less than 19 nucleotides upstream from a start codon. In certain embodiments, the target segment is less than 18 nucleotides upstream from a start codon. In certain embodiments, the target segment is less than 17 nucleotides upstream from a start codon. In certain embodiments, the target segment is less than 16 nucleotides upstream from a start codon. In certain embodiments, the target segment is less than 15 nucleotides upstream from a start codon. In certain embodiments, the target segment is less than 14 nucleotides upstream from a start codon. In certain embodiments, the target segment is less than 13 nucleotides upstream from a start codon. In certain embodiments, the target segment is less than 12 or 11 nucleotides upstream from a start codon.

In certain embodiments, the target segment has a PAM or reverse complement of the PAM in a 5′ untranslated region and the PAM is less than 20 nucleotides upstream from the start codon. In certain embodiments, the target segment is in a 5′ untranslated region and the PAM or reverse complement is less than 30 nucleotides upstream from the start codon. In certain embodiments, the target segment is in a 5′ untranslated region and the PAM or reverse complement is less than 40 nucleotides upstream from the start codon.

In certain embodiments, the target segment is in a 5′ untranslated region and the PAM or reverse complement is less than 50 nucleotides upstream from the start codon. In certain embodiments, the target segment is in a 5′ untranslated region and the PAM or reverse complement is less than 100 nucleotides upstream from the start codon. In certain embodiments, the 5′ untranslated region and the PAM or reverse complement is less than 200 nucleotides upstream from the start codon. In certain embodiments, the 5′ untranslated region and the PAM or reverse complement is less than 300 nucleotides upstream from the start codon. In certain embodiments, the 5′ untranslated region and the PAM or reverse complement is less than 400 nucleotides upstream from the start codon. In certain embodiments, the 5′ untranslated region and the PAM or reverse complement is less than 500 nucleotides upstream from the start codon. In certain embodiments, the 5′ untranslated region and the PAM or reverse complement is less than 1000 nucleotides upstream from the start codon. In certain embodiments, the 5′ untranslated region and the PAM or reverse complement is less than 1500 nucleotides upstream from the start codon. In certain embodiments, the 5′ untranslated region and the PAM or reverse complement is less than 2500 nucleotides upstream from the start codon.

In certain embodiments, this disclosure relates to in vitro or in vivo methods of repressing replication of a double stranded nucleic acid comprising mixing a Cas nuclease, which is a Cpf1, with a double stranded nucleic acid sequence comprising a target segment with a protospacer adjacent motif (PAM) sequence, wherein the PAM sequence is on the 5′ end of the target sequence, wherein mixing is with an first RNA sequence comprising a guide sequence, wherein the guide sequence is identical to the sense strand target sequence for 6 or more nucleotides but less than 18 or 17 nucleotides in length starting in the 3′ direction of the PAM sequence, and wherein the Cas nuclease specifically binds the double stranded nucleic acid repressing replication of the nucleic acid without cleaving the nucleic acid. In certain embodiments, the target sequence does not encode a protein. In certain embodiments, the target sequence is an untranslated region upstream from a start codon, such as in a promotor region.

In certain embodiments, this disclosure relates to in vitro or in vivo methods of repressing replication of a double stranded nucleic acid comprising mixing a Cas nuclease, which is a Cas9, with a double stranded nucleic acid sequence comprising a target segment with a protospacer adjacent motif (PAM) sequence, wherein the PAM sequence is on the 3′ end of the target sequence, wherein mixing is with an first RNA sequence comprising a guide sequence linked to tracrRNA, wherein the guide sequence is identical to the sense strand target sequence for 6 or more nucleotides but less than 18 or 17 nucleotides in length starting in the 5′ direction of the PAM sequence, and wherein the Cas nuclease specifically binds the double stranded nucleic acid repressing replication of the nucleic acid without cleaving the nucleic acid. In certain embodiments, the target sequence does not encode a protein. In certain embodiments, the target sequence is an untranslated region up-stream from a start codon, such as in a promotor region.

In certain embodiments, this disclosure relates to methods of cleaving a double stranded nucleic acid comprising the steps of repressing replication of a double stranded nucleic acid as disclosed herein, further comprising mixing a second guide RNA sequence comprising a guide sequence and a segment capable of hybridizing to a tracrRNA under conditions such the expression of the RNA sequence forms a complex with a tracrRNA, wherein the guide sequence is identical to the target sequence for 18 or 17 or more nucleotides and wherein a Cas nuclease cleaves at least one stranded of the double stranded nucleic acid. In certain embodiments, the method comprises cleaving both strands of the nucleic acid. In certain embodiments, the method comprises cleaving the sense strand of the nucleic acid. In certain embodiments, the method comprises cleaving the template strand of the nucleic acid.

In certain embodiments, the target sequence is in the sense strand or template strand, the Cas nuclease is Cas9 and the second guide RNA has a target sequence for 18 or 17 or more nucleotides in length starting in the 5′ direction of the PAM sequence.

In certain embodiments, the target sequence is in the sense strand or template strand, the Cas nuclease is Cpf1 and the second guide RNA has a target sequence for 18 or 17 or more nucleotides in length starting in the 3′ direction of the PAM sequence.

In certain embodiments, this disclosure relates to methods of cleaving a double stranded nucleic acid comprising the steps of repressing replication of a double stranded nucleic acid disclosed herein, further comprising inserting into the cell a second vector encoding a guide RNA sequence comprising a guide sequence under conditions such the expression of the RNA sequence forms a complex with a tracrRNA in the cell, wherein the guide sequence is identical to the target sequence for 17 or more nucleotides in length starting in the 5′ or 3′ direction of the PAM sequence or reverse complement, and wherein a Cas nuclease inside the cells cleaves at least one stranded of the double stranded nucleic acid. In certain embodiments, the method comprises cleaving both strands of the nucleic acid. In certain embodiments, the method comprises cleaving the sense strand of the nucleic acid. In certain embodiments, the method comprises cleaving the template strand of the nucleic acid.

In certain embodiments, this disclosure relates to methods of repressing replication of a double stranded nucleic acid comprising: a) providing a cell with a double stranded nucleic acid sequence comprising a protospacer adjacent motif (PAM) sequence or reverse complement adjacent to a target sequence, wherein the PAM sequence or reverse complement is on the 3′ end or 5′ end of a target sequence; b) inserting into the cell a single stranded RNA sequence optionally linked to tracrRNA and/or inserting into the cells a vector encoding a Cas nuclease, e.g., Cas9 or Cpf1, and optionally a single stranded RNA sequence comprising a guide sequence optionally linked to tracrRNA, wherein the guide sequence is identical to the target sequence for 6 or more nucleotides but less than 18 or 17 nucleotides in length adjacent to and starting in the 5′ direction or 3′ direction of the PAM sequence or reverse complement, wherein the Cas nuclease specifically binds the double stranded nucleic acid without cleaving the double stranded nucleic acid repressing replication of the nucleic acid. In certain embodiments, repressing replication of a double stranded nucleic acid is repressing RNA transcription, expression or gene expression. In certain embodiments, the RNA is mRNA or microRNA. In certain embodiments, the sense strand target sequence does not encode a protein. In certain embodiments, the sense strand target sequence is an untranslated region up-stream from a start codon, such as in a promotor region. In certain embodiments, this disclosure relates to targeting multiple sites in the same promoter region using multiple guides.

In certain embodiments, the PAM is at a distance away from the start codon that enables binding of the Cas nuclease without being dislodging by RNA polymerase from the gene. Thus, it is contemplated that a Cas nuclease binds to a promoter more generally; thus, has the ability to repress two different genes by targeting the same site in the same region.

In certain embodiments, the double stranded nucleic acid is DNA or RNA. In certain embodiments, the double stranded nucleic acid is human, viral or bacterial, DNA or RNA. In certain embodiments, repressing replication is preventing or slowing a segment of DNA or RNA from being copied into RNA by an RNA polymerase. In certain embodiments, the RNA is mRNA or microRNA.

In certain embodiments, the protospacer adjacent motif (PAM) sequence is any PAM sequence associated with a Cas9 or Cpf1.

In certain embodiments, the guide RNA sequence for repressing replication is only identical to the target coding sequence for between 7 and 16 nucleotides, or 7 and 15 nucleotides, or 8 and 16 nucleotides, or 8 and 15 nucleotides in length starting in the 5′ direction or 3′ direction of the PAM sequence or reverse complement of the PAM.

In certain embodiments, any methods disclosed herein further comprises inserting into the cell a Cas nuclease, such as Cas9 or Cpf1, or a vector encoding the Cas nuclease, such as Cas9 or Cpf1, in operable combination with a promoter. In certain embodiments, the method further comprises inserting into the cell tracrRNA or a vector encoding tracrRNA in operable combination with a promoter. In certain embodiments, the method further comprises inserting into the cell sgRNA (e.g., guide sequence linked to a crRNA or a guide sequence linked to a crRNA and linked to a tracrRNA) or a vector encoding sgRNA in operable combination with a promoter.

In certain embodiments, this disclosure relates to methods of repressing gene expression, mRNA or microRNA expression of a gene comprising: a) providing a cell with a double stranded nucleic acid sequence encoding a gene comprising a target segment with a protospacer adjacent motif (PAM) sequence or reverse complement, wherein the PAM sequence is on the 3′ end or 5′ end of the target sequence; b) inserting into the cell a single stranded gRNA sequence optionally linked to tracrRNA or a vector encoding a single stranded RNA sequence comprising a guide RNA sequence optionally linked to tracrRNA, wherein the guide RNA sequence is only identical to the target sequence for 5 or 6 or more nucleotides but less than 18 or 17 nucleotides in length starting in the 5′ direction or 3′ direction of the PAM sequence or reverse complement, wherein the guide RNA and double stranded nucleic acid forms a complex that specifically binds a Cas nuclease, such as Cas9 or Cpf1, without cleaving the double stranded nucleic acid repressing replication of the nucleic acid. In certain embodiments, the sense strand target sequence does not encode a protein. In certain embodiments, the sense strand target sequence is an untranslated region up-stream from a start codon, such as in a promotor region. In certain embodiments, to proximity to the start codon, in case there is a distant enhancer element for transcription can be up to 100 nt.

In certain embodiments, the method further comprises inserting into the cell a Cas nuclease, such as Cas9 or Cpf1, or a vector encoding the Cas nuclease, such as Cas9 or Cpf1, in operable combination with a promoter. In certain embodiments, the method further comprises inserting into the cell tracrRNA or a vector encoding tracrRNA in operable combination with a promoter.

In certain embodiments, this disclosure relates vectors encoding a Cas nuclease and RNA reported herein. In certain embodiments, the vectors contain heterologous nucleic acid sequences such that the entire vector is not naturally occurring.

In some embodiments, repressing replication of the nucleic acid inhibits transcription of at least a portion of the viral genome. A targeting sequence may be used that matches the target according to predetermined criteria and does not match any portion of a host genome.

In certain embodiments, the disclosure contemplates methods disclosed herein for multiplexing for control transcription with a cleavage/nuclease active protein, e.g., Cpf1/Cas9.

In certain embodiments, multiplexing is accomplished by using separately encoded guide RNAs or multiple guides encoded within a single CRISPR array

In certain embodiments, multiplexing is accomplished by Cas9/Cpf1 for nucleic acid detection and transcriptional control, and for active Cas9/Cpf1 for cleaving different nucleic acid substrates wherein same Cas nuclease performs these functions. In certain embodiments, multiplexing is accomplished by cleavage-capable, active Cas nucleases to control transcription with a partial guide sequence.

In certain embodiments, the disclosure contemplates methods of using multiple Cas nucleases (cas9 and cpf1) simultaneously with each system programmed for multiple functions such as cleavage and transcriptional modification.

In certain embodiments, the disclosure contemplates methods of targeting bacteria. In certain embodiments, the disclosure contemplates methods of targeting mobile genetic element interactions e.g., repress phage genes vs cleaving some of them. In certain embodiments, the disclosure contemplates methods of enhancing expression of a genomic locus that encodes genes to help phages replicate, or plasmids replicate/conjugate, while also cleaving incoming DNA. In certain embodiments, the disclosure contemplates methods of suppressing homologous recombination with the CRISPR system to prevent survival of strains with the prophage if a prophage integrates and the bacteria cleaves its own chromosome,

In certain embodiments, Cas9 nucleases are capable of ssRNA cleavage and ssDNA cleavage. Cpf1 can mediate ssDNA cleavage. In certain embodiments, cleavage is guide RNA independent. In certain embodiments, ssRNA cleavage by a Cas nuclease could be employed to target specific splicing products from mammalian RNAs in tandem with partial guide RNAs repressing or activating transcription to more comprehensively reduce RNA levels without modifying the genome.

In certain embodiments, Cas nucleases orthologs can degrade ssDNA or ssRNA. Thus, in certain embodiments, this disclosure contemplates multiplexing the cleavage and repression functions with guide RNAs that also guide these functions. In certain embodiments, this disclosure contemplates using Cas nucleases to bind but not cleave with a partial RNA can also be utilized to detect nucleic acids (another function the same Cas nuclease can be used for based on its RNA guide, and thus can be multiplexed for) e.g., dCas9 or active Cas9 with a partial guide RNA.

### BRIEF DESCRIPTION OF THE DRAWINGS

FIG. 1A illustrates that FnoCas9 (Francisella novicida Cas9) uses scaRNA to bind endogenous DNA and repress transcription. The limited length of scaRNA:target complementarity prevents DNA cleavage. Cleavage-competent FnoCas9 uses distinct RNAs for repression versus cleavage. Regulation occurs through a protospacer adjacent motif (PAM)-dependent interaction of Cas9 with its endogenous DNA targets, dependent on a non-canonical small RNA (scaRNA) and tracrRNA. Experiments indicate that the scaRNA can be reprogrammed to guide FnoCas9 to repress a new target.

FIG. 1B illustrates the genomic locus encoding the four genes that are repressed by Cas9, tracrRNA and scaRNA. These genes are encoded on two distinct mRNA transcripts. Each of these transcripts contains 5′ and 3′ untranslated regions (UTRs) that do not encode for gene products. The boxes represent the location on the DNA and the mRNA of the 5′ UTRs of the 1104-1102 and 1101 transcripts (38 base pairs (bp) long and 72 base pairs long (bp) respectively.)

FIG. 2A illustrates promoter fusion constructs with combinations of the 1104 promoter, 1104 5′ UTR, and a synthetic promoter indicate that Cas9 repressed transcript level with the 5′ UTR. The alternative promoter was a synthetic promoter identified to be constitutively active in Francisella. The promoter and UTRs were fused to a fragment of GFP derived from the pBav plasmid vector. The arrow indicates the transcriptional start site for each fusion construct.

FIG. 2B shows transcript levels of the non-native gfp transcript by quantitative real-time PCR (qRTPCR) is presented as relative transcript in each strain relative to the housekeeping gene in F. novicida uvrD that is unaffected by the fusion constructs. The 5′ UTR alone is enough to enable Cas9 to repress gfp transcripts from the synthetic promoter.

FIG. 2C illustrates promoter fusion constructs with combinations of the 1101 promoter, 1101 5′ UTR, and a synthetic promoter indicate that Cas9 repressed transcript level with the 5′ UTR.

FIG. 2D shows relative transcript level of gfp was determined.

FIG. 3A illustrates DNA recognition and binding by the Cas9: tracrRNA-scaRNA (SEQ ID NOs: 7-8) complex for the 1104 5′ UTR (SEQ ID NO: 5). tracrRNA: scaRNA in complex with Cas9, with interaction between scaRNA (SEQ ID NO: 7) and 1104 3′ UTR (SEQ ID NO: 6) depicted. PAM sequence is indicated in with a box.

FIG. 3B illustrates the interaction of tracrRNA-scaRNA (SEQ ID NOs: 7-8) with 1101 5′ UTR (SEQ ID NO: 9) and 3′ UTR (SEQ ID NO: 10).

FIG. 3C shows data on the effect of PAM mutations on the ability of Cas9 to repress transcription. Mutation of the nGG pam to a nAA fully prevents Cas9 from repressing transcription.

FIG. 3D shows data on PCR analysis of the RNA bound to wild-type Cas9 and a Cas9:R59A mutant that is unable to bind tracrRNA and scaRNA. The wild-type Cas9 has a much stronger interaction with the 1104 and 1101 promoter regions while the two Cas9 variants have the same interaction with a control sequence elsewhere in the bacterial genome.

FIG. 4A shows data indicating Cpf1 represses transcription from a plasmid using a partial crRNA sequence that is unable to inhibit transformation with the plasmid. Plasmid constructs were designed with gfp under the control of a synthetic constitutive promoter for Francisella novicida. Between the transcriptional start site of the promoter and gfp, the F. novicida Cpf1 PAM (FnoCpf1, protospacer adjacent motif) was followed by 0, 8, 11, 15, 20 or 29 bases of complementarity to a native FnoCpf1 crRNA spacer. 29 bp is the full length of the native spacer. These constructs were transformed into wild-type (WT) and a cpf1 mutant of F. novicida and the ability of WT to restrict transformation with each construct was enumerated as % transformants recovered from WT relative to a cpf1 mutant. Constructs with 0, 8, 11, and 15 bp of crRNA spacer identity transformed with the same efficiency into both WT and a cpf1 mutant. However, Cpf1 was able to restrict transformation into WT when the plasmid contained 20 and 29 bases of identity to crRNA.

FIG. 4B shows data where transformants were cultured successful from WT and cfp1 containing either the 0, 8, 11, and 15 bp plasmids. Measured the level of gfp mRNA by qRT-PCR are enumerated in as % gfp transcripts recovered from WT relative to a cpf1 mutant. gfp expression from the plasmid with 15 bases of complementarity to a Cpf1 spacer is reduced, suggesting that the partial crRNA is guiding Cpf1 to bind but not to cleave at the promoter of the plasmid.

FIG. 4C shows data indicating FnoCas9 transcriptional interference is controlled by target proximity to the transcriptional start site (TSS). An 11 bp sequence with complementarity to the scaRNA tail and adjacent to a PAM was placed downstream of a synthetic constitutive promoter, driving the expression of gfp* (+/−Cas9). A plasmid with gfp* was placed downstream of a synthetic promoter, and 0 bp of identity between scaRNA and the TSS region of the plasmid was used as a control. Relative gfp transcript levels were measured by qRT-PCR in strains with varying numbers of additional bases (0 to 20 bp) placed between the TSS and the sequence with complementarity to the scaRNA.

FIG. 5 shows data indicating FnoCas9 transcriptional interference is controlled by target proximity to the TSS. An 11 bp sequence with complementarity to the scaRNA tail and adjacent to a PAM was placed downstream of a synthetic constitutive promoter, driving the expression of gfp* (+/−Cas9). A plasmid with gfp* was placed downstream of a synthetic promoter, and 0 bp of identity between scaRNA and the TSS region of the plasmid was used as a control. Relative gfp transcript levels were measured by qRT-PCR in strains with varying numbers of additional bases (0 to 20 bp) placed between the TSS and the sequence with complementarity to the scaRNA.

FIG. 6A shows a schematic of the scaRNA target site when reprogrammed for the 98 bp intergenic region between two F. novicida virulence factors, 0544 and 0545, that are transcribed in opposite directions.

FIG. 6B shows qRT-PCR for transcript levels of 0544 in WT, scaRNA_0544/0545 (WT with the scaRNA tail reprogrammed to target 0544/0545), and Δcas9+scaRNA_0544/0545.

FIG. 6C shows qRT-PCR for transcript levels of 0545.

FIG. 6D shows qRT-PCR for transcript levels of 1104.

FIG. 6E shows percent survival of WT, reprogrammed scaRNA, delta 0544, and delta cas9+reprogrammed scaRNA strains 6 h after polymyxin treatment (100 mg/mL) relative to untreated strains.

FIG. 7A shows data on % transformants recovered from WT relative to a cpf1 mutant.

FIG. 7B shows data as % gfp transcripts recovered from WT relative to a cpf1 mutant.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. patent law in that they are inclusive or open-ended and do not exclude additional, unrecited elements or method steps. The term “comprising” in reference to an oligonucleotide having a nucleic acid sequence refers to an oligonucleotide that may contain additional 5′ (5′ terminal end) or 3′ (3′ terminal end) nucleotides, i.e., the term is intended to include the oligonucleotide sequence within a larger nucleic acid. “Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein. The term “consisting of” in reference to an oligonucleotide having a nucleotide sequence refers an oligonucleotide having the exact number of nucleotides in the sequence and not more or having not more than a range of nucleotide expressly specified in the claim. For example, “5′ sequence consisting of” is limited only to the 5′ end, i.e., the 3′ end may contain additional nucleotides. Similarly, a “3′ sequence consisting of” is limited only to the 3′ end, and the 5′ end may contain additional nucleotides.

“Sequence identity” refers to a measure of relatedness between two or more nucleic acids or proteins, and is typically given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.) using default parameters. In certain embodiments, sequence “identity” refers to the number of exactly matching residues (expressed as a percentage) in a sequence alignment between two sequences of the alignment. In certain embodiments, percentage identity of an alignment may be calculated using the number of identical positions divided by the greater of the shortest sequence or the number of equivalent positions excluding overhangs wherein internal gaps are counted as an equivalent position. For example, the polypeptides GGGGGG (SEQ ID NO: 20) and GGGGT (SEQ ID NO: 21) have a sequence identity of 4 out of 5 or 80%. For example, the polypeptides GGGPPP (SEQ ID NO: 22) and GGGAPPP (SEQ ID NO: 23) have a sequence identity of 6 out of 7 or 85%.

In certain embodiments, for any contemplated percentage sequence identity, it is also contemplated that the sequence may have the same percentage of sequence similarity. Percent “similarity” is used to quantify the extent of similarity, e.g., hydrophobicity, hydrogen bonding potential, electrostatic charge, of amino acids between two sequences of the alignment. This method is similar to determining the identity except that certain amino acids do not have to be identical to have a match. In certain embodiments, sequence similarity may be calculated with well-known computer programs using default parameters. Typically, amino acids are classified as matches if they are among a group with similar properties, e.g., according to the following amino acid groups: Aromatic—F Y W; hydrophobic-A V I L; Charged positive: R K H; Charged negative—D E; Polar—S T N Q.

A partially complementary sequence is one that at least partially inhibits (or competes with) a completely complementary sequence from hybridizing to a target nucleic acid—also referred to as “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. In certain embodiment, the term “sequence identity” refers to two polynucleotide sequences are identical (i.e., on a nucleotide-by-nucleotide basis) over the window of comparison. In some embodiments, the term “percentage of sequence identity” over a comparison window is calculated by comparing two optimally aligned sequences over the window of comparison, determining the number of positions at which the identical nucleic acid base (e.g., A, T/U, C, G, or I) occurs in both sequences to yield the number of matched positions, dividing the number of matched positions by the total number of positions in the window of comparison (i.e., the window size), and multiplying the result by 100 to yield the percentage of sequence identity.

The terms, the “sense strand” refers to the coding strand, and the antisense “template strand” refers to as a non-coding strand, whether or not the sense strand target segment or template strand target segment is actually encoding a protein. The target sequence is intended to include untranslated regions, promoter regions, etc. During transcription, a RNA polymerase binds the template strand of double stranded DNA or RNA, reads the anti-codons, and transcribes their sequence to synthesize an RNA transcript with complementary bases. It is also understood that RNA contains uracil (U) and DNA sequences contain thymine (T). Thus, in this context, T and U are considered identical despite the fact that from a molecular structure T includes a methyl group that U lacks.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides.

Nucleic acids of the present disclosure may also be synthesized, either completely or in part, especially where it is desirable to provide plant-preferred sequences, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The term “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The sense strand is the segment within double-stranded DNA that runs from 5′ to 3′, and which is complementary to the template strand, which runs from 3′ to 5′. The sense strand is the strand of DNA that has the same sequence as the mRNA, which takes the template strand as its template during transcription.

The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene. The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into a nucleic acid of another species or of synthetic origin. A heterologous gene also includes a gene native to an organism that has been altered in some way (e.g., mutated, added in multiple copies, linked to a non-native promoter or enhancer sequence, etc.). Heterologous genes may comprise bacterial gene sequences that comprise cDNA forms of a bacterial gene; the cDNA sequences may be expressed in either a sense (to produce mRNA) or anti-sense orientation (to produce an anti-sense RNA transcript that is complementary to the mRNA transcript).

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules, or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The nucleic acid molecules or guided or targeting RNA disclosed herein are capable of specifically hybridizing to the target nucleic acid under certain circumstances. As used herein, two nucleic acid molecules are said to be capable of specifically hybridizing to one another if the two molecules are capable of forming a hydrogen bonding nucleic acid structure. A nucleic acid molecule may exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook, et al. (1989), and by Haymes et al. (1985).

Departures from complete complementarity are therefore permissible, as long as such departures do not completely preclude the capacity of the RNA molecules to form a hydrogen bonding structure with the target. Thus, in order for an RNA to serve as a guide to the target, the RNA needs only be sufficiently complementary in sequence to be able to form a stable hydrogen bonding structure under the physiological conditions of the cell expressing the RNA.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

A “vector” refers to a nucleic acid molecule used as a vehicle to carry foreign genetic material into cell, where it can be replicated and/or expressed. A cloning vector containing foreign nucleic acid is termed a recombinant vector. Examples of vectors are plasmids, viral vectors, cosmids, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs) are for the expression of the transgene in the target cell, and generally have a promoter sequence that drives expression of the transgene. Insertion of a vector into the target cell is referred to transformation or transfection for bacterial and eukaryotic cells, although insertion of a viral vector is often called transduction.

In certain embodiments, a vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env. In certain embodiments, the vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter, e.g., heterologous because the promoter and the gene are from different organisms. For example, an endogenous promoter in operable combination with a first gene can be isolated, removed, and placed in operable combination with a second gene, thereby making it a “heterologous promoter” in operable combination with the second gene as they do not naturally occur together in nature.

Efficient expression of recombinant DNA sequences in eukaryotic cells is believed to include the expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene.

Efficient expression of recombinant DNA sequences in eukaryotic cells is believed to include the expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene.

A “selectable marker” is a nucleic acid introduced into a vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: ampr, camr, tetr, blasticidinr, neor, hygr, abxr, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (at1D), UDP-glucose:galactose-1-phosphate uridyltransferasel (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

### CRISPR and Cas Nucleases

The clustered regularly interspaced short palindromic repeat (CRISPR) system is a prokaryotic adaptive immune system that has been modified to enable general genome engineering in a variety of organisms and cell lines. CRISPR-Cas (CRISPR associated) systems are protein-RNA complexes that use an RNA molecule (crRNA) as a guide (gRNA segment) to localize the complex to a target nucleic acid sequence via base-pairing.

In the natural systems, a CRISPR associated (Cas) proteins then acts as a nuclease to cleave the targeted DNA sequence. The target sequence contains a “protospacer-adjacent motif” (PAM) oligonucleotide adjacent to the target region in order for the system to function. Among the known Cas nucleases, such as Cas9, S. pyogenes Cas9 has been widely reported. However, other CRISPR-associated nucleases have been reported such as Cpf1 (also known as Cas12A). See e.g., WO 2017/015015 and GenBank accession number WP_003034647 is reported as a 1300 amino acid protein from Francisella novicida. GenBank accession number AJI56734.1 is reported as a 939 CRISPR-associated protein from Francisella philomiragia (Cpf1).

For Cpf1 is a single RNA-guided endonuclease lacks tracrRNA. Unlike Cas9 systems, Cpf1-associated CRISPR arrays are processed into mature crRNAs without the requirement of an additional trans-activating crRNA (tracrRNA). Cpf1-crRNA complexes efficiently cleave target DNA proceeded by a short T-rich protospacer-adjacent motif (PAM), in contrast to the G-rich PAM following the target DNA for Cas9 systems, and the target sequence is typically in the 3′ direction (downstream) from the PAM sequence, in contrast the target DNA for Cas9 systems is typically in the 5′ direction (upstream) from the PAM sequence. Cpf1 introduces a staggered DNA double-stranded break with a 4 or 5-nt 5′ overhang. The seed region of Cpf1 guide RNA is approximately within the first 5 nt on the 5′ end of the target sequence, thereafter complete complementarity is typically not critical. If Cpf1 is used for embodiments of this disclosure, the PAM is typically located with a different orientation to the protospacer than Cas9. The orientation of the Cpf1 PAM relative to 5′ and 3′ ends of the crRNA is distinction from Cas9. See Zetsche et al. Cpf1 is a single RNA-guided endonuclease of a class 2 CRISPR-Cas system. Cell, 2015, 163 (3): 759-71.

As used herein, a “guide RNA” refers to a single RNA or multiple RNAs that complex with each other containing a guide sequence for a target and other sequences that facilitate binding to a Cas nuclease.

Cas nucleases are typically large, multi-domain proteins containing two distinct nuclease domains. Point mutations can be introduced into Cas nucleases to abolish nuclease activity, resulting in a nuclease inactive Cas nuclease, such as Cas9 and Cpf1, that still retains its ability to bind DNA in a gRNA-programmed manner. By creating Cas nuclease fusion proteins with protein domains one can alter the rate of gene translation into mRNA, e.g., transcription factors and regulators, the CRISPR-cas system functions as a RNA guided gene expression controller.

Wild-type Cas9 proteins have two functional endonuclease domains, RuvC and HNH. The RuvC domain cleaves one strand of a double strand DNA and the HNH domain cleaves another strand. When the both domains are active, the Cas9 protein can generate the double strand breaks in genomic DNA. Cas9 proteins having only one of the enzymatic activities have been developed. Such Cas9 proteins cleave only one strand of the target DNA. For example, the RuvC and HNH domains of the Cas9 protein derived from Streptococcus pyogenes are inactivated by D10A and H840A mutations, respectively.

Ability of Cas9 proteins to bind to a target DNA is independent from their ability to cleave the target DNA. Even if both of the RuvC and HNH domains are inactive and the Cas9 protein has no nuclease activity, the Cas9 protein still retains the ability to bind to the target DNA in the presence of gRNA. Accordingly, Cas9 proteins lacking nuclease activity (dCas9 proteins) may be used as a tool in molecular biology. For example, such dCas9 proteins may be used as a transcriptional regulator to activate or suppress expression of a gene through binding to a known transcriptional regulatory domain via gRNA. For example, if a dCas9 protein is fused with a transcriptional activator, it can activate transcription of the target gene. To the contrary, when only the dCas9 protein binds to the target sequence, the transcription may be suppressed. Expression of various genes may be regulated by targeting a sequence close to the promoter of the desired gene. Alternatively, in assays such as chromatin immunoprecipitation, genomic DNA may be purified by using a dCas9 protein fused with an epitope tag and a gRNA that targets any sequence in the genomic DNA. When a dCas9 protein fused with a fluorescent protein such as GFP or mcherry is used together with a gRNA that targets a desired sequence in genomic DNA, it may be used as a DNA label that can be detected in a living cell. Contemplated herein are similar methods wherein dCas9 can be substituted for an active Cas nuclease and a shortened guide RNA sequence.

As used herein, the term “Cas nucleases” means a protein having an ability to bind to a DNA molecule in the presence of gRNA, including Cpf1 and Cas9 proteins, having dual nuclease activities, e.g., both the RuvC and HNH nuclease activities for Cas9 proteins and both a RuvC-like endonuclease and a second nuclease domain for Cpf1, or lacking either or both the two nuclease activities. The DNA-binding activity and nuclease activity of Cas nucleases are described in Sternberg et al., Nature, 507, 62-67 (2014).

In certain embodiments, a Cas nuclease derived from a bacterium having a CRISPR system is used. Bacteria known to have a CRISPR system include bacteria belonging to Aeropyrum sp., Pyrobaculum sp., Sulfolobus sp., Archaeoglobus sp., Halocarcula sp., Methanobacteriumn sp., Methanococcus sp., Methanosarcina sp., Methanopyrus sp., Pyrococcus sp., Picrophilus sp., Thermoplasma sp., Corynebacterium sp., Mycobacterium sp., Streptomyces sp., Aquifex sp., Porphyromonas sp., Chlorobium sp., Thermus sp., Bacillus sp., Listeria sp., Staphylococcus sp., Clostridium sp., Thermoanaerobacter sp., Mycoplasma sp., Fusobacterium sp., Azoarcus sp., Chromobacterium sp., Neisseria sp., Nitrosomonas sp., Desulfovibrio sp., Geobacter sp., Micrococcus sp., Campylobacter sp., Wolinella sp., Acinetobacter sp., Erwinia sp., Escherichia sp., Legionella sp., Methylococcus sp., Pasteurella sp., Photobacterium sp., Salmonella sp., Xanthomonas sp., Yersinia sp., Treponema sp., and Thermotoga sp. For example, a Cas9 protein derived from a bacterium such as Streptococcus pyogenes, Neisseria meningitides, Streptococcus thermophiles, or Treponema denticola is used.

In certain embodiment, a Cas nuclease which is a fusion protein with at least one other protein or peptide may be used. Such proteins and peptides include, for example, fluorescent proteins, transcriptional factors, epitope tags, tags for protein purification, nuclear localization signal peptides, and transcriptional regulators such as an activator or repressor. In certain embodiments, it is contemplated that that the Cas nuclease is not fused to a transcriptional regulator.

Cas nucleases mRNA may be obtained by cloning a DNA coding an amino acid sequence of a desired Cas nuclease into a vector suitable for in vitro transcription and performing in vitro transcription. Vectors suitable for in vitro transcription are known to those skilled in the art. In vitro transcription vectors that contain a cloned DNA encoding a Cas9 protein are also known and include, for example, pT7-Cas9 available from Origene. Methods of in vitro transcription are known to those skilled in the art.

In certain embodiments, a solution comprising Cas nucleases mRNA may contain at least one further nucleic acid and the nucleic acid may be introduced to a cell together with the mRNA.

The further nucleic acid may be, for example, gRNA, scaRNA, crRNA, tracrRNA or sgRNA. For example, gRNA alone, combination of scaRNA and tracrRNA or crRNA and tracrRNA, combination of gRNA and sgRNA, or combination of scaRNA, crRNA, tracrRNA and sgRNA may be used.

Guide RNAs generally speaking comes in different forms. One form uses separate targeting guide RNA and a tracrRNA that hybridize together to guide targeting, and another, which uses a chimeric targeting guide RNA-tracrRNA hybrid that links the two separate RNAs in a single strand of RNA that forms a hairpin, referred to as sgRNA. See also Jinek et al., Science 2012; 337:816-821. The tracrRNA can be variably truncated and a range of lengths has been shown to function in both forms, separate hybridizing segments and the chimeric sgRNA. For example, in some embodiments, tracrRNA may be truncated from its 3′ end by at least 1, 2, 3, 4, 5, 6, 7, 8, 9, or 10 nts. In some embodiments, the tracrRNA molecule may be truncated from its 5′ end by at least 1, 2, 3, 4, 5, 6, 7, 8, 9, or 10 nts. Alternatively, the tracrRNA molecule may be truncated from both the 5′ and 3′ end, e.g., by at least 1, 2, 3, 4, 5, 6, 7, 8, 9, or 10 nts on the 5′ end and at least 1, 2, 3, 4, 5, 6, 7, 8, 9, or 10 nts on the 3′ end.

In the natural state, crRNA is responsible for sequence specificity of gRNA. The target sequence may be present in either strand of the genomic DNA. However, in a preferred embodiment of this disclosure, the gRNA comprises a sequence that is identical to the sense or template strand that is upstream or downstream from the PAM or reverse complement of the PAM. Tools are available for selecting a target sequence and/or designing gRNA, and lists of target sequences which are predicted for various genes in various species may be obtained. For example, Feng Zhang lab's Target Finder, Michael Boutros lab's Target Finder (E-CRISP), RGEN Tools: Cas-OFFinder, CasFinder: Flexible algorithm for identifying specific Cas9 targets in genomes, and CRISPR Optimal Target Finder, may be mentioned and the entire contents thereof are incorporated herein by reference.

Cas nucleases can bind to any DNA that has the PAM sequence. The exact sequence of the PAM is dependent upon the bacterial species from which the Cas nuclease is derived. One Cas9 protein is derived from Streptococcus pyogenes and the corresponding PAM sequence is NGG present immediately downstream of the 3′ end of the target sequence. PAM sequences of various bacterial species are known, for example, Neisseria meningitides: NNNNGATT (SEQ ID NO: 2), Streptococcus thermophiles: NNAGAA (SEQ ID NO: 3), Treponema denticola: NAAAAC (SEQ ID NO: 4). In these sequences, N represents any one of A, T/U, G, and C.

In bacteria, tracrRNA hybridizes to a part of gRNA to form a hairpin loop structure. The structure is recognized by Cas9 protein and a complex of crRNA, tracrRNA and Cas9 protein is formed. Thus, tracrRNA is responsible for the ability of gRNA to bind to Cas9 protein. TracrRNA is derived from an endogenous bacterial RNA and has a sequence intrinsic to the bacterial species. TracrRNA derived from the bacterial species known to have a CRISPR system listed above may be used herein. Preferably, tracrRNA and Cas9 protein derived from the same species are used. For example, tracrRNA derived from Streptococcus pyogenes, Neisseria meningitides, Streptococcus thermophiles, or Treponema denticola may be used.

Guide RNA (gRNA) may be obtained by cloning a DNA having a desired gRNA sequence into a vector suitable for in vitro transcription and performing in vitro transcription. Vectors suitable for in vitro transcription are known to those skilled in the art. In vitro transcription vectors that comprise a sequence corresponding to gRNA with no target sequence are also known in the art. Guide RNA may be obtained by inserting a synthesized oligonucleotide of a target sequence into such vector and performing in vitro transcription. Such vectors include, for example, pUC57-sgRNA expression vector, pCFD1-dU6:1gRNA, pCFD2-dU6:2gRNA pCFD3-dU6:3gRNA, pCFD4-U6:1_U6:3tandemgRNAs, pRB17, pMB60, DR274, SP6-sgRNA-scaffold, pT7-gRNA, DR274, and pUC57-Simple-gRNA backbone available from Addgene, and pT7-Guide-IVT available from Origene. Methods of in vitro transcription are known to those skilled in the art.

Combination of a guide sequence linked to a crRNA and separate tracrRNA may be used in place of a gsRNA. When the combination is used, the crRNA and tracrRNA are separate RNA molecules.

In certain embodiments, the disclosure relates to methods of modifying eukaryotic cells by manipulation of a target sequence in a genomic locus of interest comprising delivering a non-naturally occurring or engineered vector or one or more vectors operably encoding systems herein discussed for expression thereof.

In certain embodiments, the eukaryotic cell is a stem cell, a somatic cell, differentiated somatic cell, a reprogrammed induced pluripotent somatic stem cell, reprogrammed induced pluripotent somatic stem cell.

In certain embodiments, the somatic cell is selected from the group consisting of a mesenchymal somatic cell, a fibroblast somatic cell, a cardiomyocyte somatic cell, a hematopoietic cell, and a pancreatic beta somatic cell. In one embodiment, said somatic cell is selected from the group consisting of a mesenchymal somatic cell, a fibroblast somatic cell, a cardiomyocyte somatic cell, a hematopoietic cell, and a pancreatic beta somatic cell. In one embodiment, the differentiated somatic cell is selected from the group consisting of a fibroblast cell. In one embodiment, the reprogrammed induced pluripotent somatic stem cell is selected from the group consisting of a neuronal cell, a motor neuron cell, a cortical neuron cell and an astrocyte cell. In one embodiment, the reprogrammed induced pluripotent somatic stem cell is selected from the group consisting of a mesenchymal somatic cell, a fibroblast somatic cell, a cardiomyocyte somatic cell, a hematopoietic cell and a pancreatic beta somatic cell. In one embodiment, the reprogrammed induced pluripotent somatic stem cell is selected from the group consisting of a pancreatic endocrine cell, a cardiomyocyte cell, a thymic epithelial cell and a thyroid cell. In one embodiment, the regulating transcription of said specific genomic target results in a phenotypic change of said reprogrammed induced pluripotent somatic stem cell.

In some embodiments, the nucleic acid encoding Cas nuclease is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available.

In some embodiments, a vector encodes Cas nuclease comprising one or more nuclear localization sequences (NLSs). In some embodiments, the CRISPR enzyme comprises about or more than about 1 or more NLSs at or near the amino-terminus, about or more than about 1 or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Typically, NLS consists of one or more short sequences of positively charged lysines or arginines exposed on the protein surface, but other types of NLS are known.

In certain embodiments, this disclosure relates to compositions comprising one or more nucleic acids as described herein and a pharmaceutically acceptable excipient. In certain embodiments, a pharmaceutically acceptable excipient selected from the group consisting of a solvent, aqueous solvent, non-aqueous solvent, dispersion media, diluent, dispersion, suspension aid, surface-active agent, isotonic agent, thickening or emulsifying agent, preservative, lipid, lipidoids, liposomes, lipid nanoparticles, core-shell nanoparticles, polymer, lipoplex, peptide, protein, cell, hyaluronidase, and mixtures thereof.

In certain embodiments, this disclosure relates to synthetic sgRNAs for targeting a gene of interest, the sgRNA comprising a guide sequence identical to the sense strand target sequence for 6 or more nucleotides but less than 17 nucleotides in length starting in the 5′ direction of the PAM sequence.

In certain embodiments the sgRNA comprises at least one 5′ cap structure, first modified nucleoside, or a modifications located on one or more of a nucleoside and/or a backbone linkage between nucleosides or at least one or two modifications are located on both a nucleoside and a backbone linkage; or at least one or two modification is located on a backbone linkage; or at least one or two or more backbone linkages are modified by replacement of one or more oxygen atoms or at least one or two modification comprises replacing at least one backbone linkage with a phosphorothioate linkage or at least one or two modification is located on one or more nucleosides or at least one or two one or more modifications are on a sugar of one or more nucleosides or at least one or two or at least one modification is located on one or more nucleobases selected from the group consisting of cytosine, guanine, adenine, thymine and uracil. In certain embodiments, the modification is a non-translating modification, e.g., increases stabilization in serum.

Cas9 proteins contain four RuvC endonuclease domains (RuvC-I through RuvC-IV), as well as an HNH endonuclease domain. RuvC-I and the HNH necessary for degradation of target DNA. Cas9 also contains an arginine-rich motif (ARM). Francisella novicida U112 Cas9 has the amino acid sequence:

(SEQ ID NO: 24)
MNFKILPIAIDLGVKNTGVFSAFYQKGTSLERLDNKNGKVYELSKDSY
TLLMNNRTARRHQRRGIDRKQLVKRLFKLIWTEQLNLEWDKDTQQAIS
FLFNRRGFSFITDGYSPEYLNIVPEQVKAILMDIFDDYNGEDDLDSYL
KLATEQESKISEIYNKLMQKILEFKLMKLCTDIKDDKVSTKTLKEITS
YEFELLADYLANYSESLKTQKFSYTDKQGNLKELSYYHHDKYNIQEFL
KRHATINDRILDTLLTDDLDIWNFNFEKFDFDKNEEKLQNQEDKDHIQ
AHLHHFVFAVNKIKSEMASGGRHRSQYFQEITNVLDENNHQEGYLKNF
CENLHNKKYSNLSVKNLVNLIGNLSNLELKPLRKYFNDKIHAKADHWD
EQKFTETYCHWILGEWRVGVKDQDKKDGAKYSYKDLCNELKQKVTKAG
LVDFLLELDPCRTIPPYLDNNNRKPPKCQSLILNPKFLDNQYPNWQQY
LQELKKLQSIQNYLDSFETDLKVLKSSKDQPYFVEYKSSNQQIASGQR
DYKDLDARILQFIFDRVKASDELLLNEIYFQAKKLKQKASSELEKLES
SKKLDEVIANSQLSQILKSQHTNGIFEQGTFLHLVCKYYKQRQRARDS
RLYIMPEYRYDKKLHKYNNTGRFDDDNQLLTYCNHKPRQKRYQLLNDL
AGVLQVSPNFLKDKIGSDDDLFISKWLVEHIRGFKKACEDSLKIQKDN
RGLLNHKINIARNTKGKCEKEIFNLICKIEGSEDKKGNYKHGLAYELG
VLLFGEPNEASKPEFDRKIKKENSIYSFAQIQQIAFAERKGNANTCAV
CSADNAHRMQQIKITEPVEDNKDKIILSAKAQRLPAIPTRIVDGAVKK
MATILAKNIVDDNWQNIKQVLSAKHQLHIPIITESNAFEFEPALADVK
GKSLKDRRKKALERISPENIFKDKNNRIKEFAKGISAYSGANLTDGDF
DGAKEELDHIIPRSHKKYGTLNDEANLICVTRGDNKNKGNRIFCLRDL
ADNYKLKQFETTDDLEIEKKIADTIWDANKKDFKFGNYRSFINLTPQE
QKAFRHALFLADENPIKQAVIRAINNRNRTFVNGTQRYFAEVLANNIY
LRAKKENLNTDKISFDYFGIPTIGNGRGIAEIRQLYEKVDSDIQAYAK
GDKPQASYSHLIDAMLAFCIAADEHRNDGSIGLEIDKNYSLYPLDKNT
GEVFTKDIFSQIKITDNEFSDKKLVRKKAIEGENTHRQMTRDGIYAEN
YLPILIHKELNEVRKGYTWKNSEEIKIFKGKKYDIQQLNNLVYCLKFV
DKPISIDIQISTLEELRNILTTNNIAATAEYYYINLKTQKLHEYYIEN
YNTALGYKKYSKEMEFLRSLAYRSERVKIKSIDDVKQVLDKDSNFIIG
KITLPFKKEWQRLYREWQNTTIKDDYEFLKSFFNVKSITKLHKKVRKD
FSLPISTNEGKFLVKRKTWDNNFIYQILNDSDSRADGTKPFIPAFDIS
KNEIVEAIIDSFTSKNIFWLPKNIELQKVDNKNIFAIDTSKWFEVETP
SDLRDIGIATIQYKIDNNSRPKVRVKLDYVIDDDSKINYFMNHSLLKS
RYPDKVLEILKQSTIIEFESSGFNKTIKEMLGMKLAGIYNETSNN.

In certain embodiments, this disclosure contemplates a Cas9 comprising SEQ ID NO: 24 or variants or conserved variants thereof. In certain embodiments, Cas9 has SEQ ID NO: 24 or a variant with 10%, 30%, 50%, 60%, 70%, 80%, 90%, 95%, 98% or more identity thereto.

In certain embodiments, the variants are or are not in the RuvC-I, arginine rich, RuvC-II, RuvC-III, HNH, or RuvC-IV motifs. In certain embodiments, the variants are conserved substitutions inside or outside of the RuvC-I, arginine rich, RuvC-II, RuvC-III, HNH, or RuvC-IV motifs.

In certain embodiments, the Cas9 has a RuvC-I motif has greater than about 10%, 20%, 30%, 40%, 50%, 60%, 80%, 90%, or 95% identity to PIAIDLGVKNTGVFSAFYQK (SEQ ID NO: 25).

In certain embodiments, the Cas9 has an arginine rich motif has greater than about 10%, 20%, 30%, 40%, 50%, 60%, 80%, 90%, or 95% identity to MNNRTARRHQRRGIDRKQLVK (SEQ ID NO: 26).

In certain embodiments, the Cas9 has a RuvC-II motif with greater than about 10%, 20%, 30%, 40%, 50%, 60%, 80%, 90%, or 95% identity to EWDKDTQQAISFLFNRRGFSFITDGYSPEYLNIV (SEQ ID NO: 27).

In certain embodiments, the Cas9 has a RuvC-III motif with greater than about 10%, 20%, 30%, 40%, 50%, 60%, 80%, 90%, or 95% identity to 5 KNIVDDNWQNIKQVLSAKHQLHIPIITESNAFEFE (SEQ ID NO: 28).

In certain embodiments, the Cas9 has an HNH motif with greater than about 10%, 20%, 30%, 40%, 50%, 60%, 80%, 90%, or 95% identity to LDHIIPRSHKKYGTLNDEANLICVTRGDNKNKGNRI (SEQ ID NO: 29).

In certain embodiments, the Cas9 has a RuvC-IV motif with greater than about 10%, 20%, 30%, 40%, 50%, 60%, 80%, 90%, or 95% identity to AKGDKPQASYSHLIDAMLAFCIAADEHRNDG (SEQ ID NO: 30).

In certain embodiments, conserved substitutions include amino acids with aliphatic side chains such as alanine, isoleucine, leucine, proline and valine. In certain embodiments, conserved substitutions include amino acids methionine, isoleucine, leucine, and valine. In certain embodiments, conserved substitutions include amino acids that contain two or more aliphatic carbons that connect the protein backbone such as arginine, lysine, glutamate and glutamine. In certain embodiments, conserved substitutions include amino acids that contain an aromatic side chain such as phenylalanine, tryptophan, tyrosine and histidine. In certain embodiments, conserved substitutions include amino acids that are negatively charged at typical biological pHs such as aspartate and glutamate. In certain embodiments, conserved substitutions include amino acids that are positively charged at typical biological pHs such as lysine and arginine. In certain embodiments, conserved substitutions include amino acids that are neutral at typical biological pHs but are polar such as histidine, asparagine, glutamine, serine, threonine and tyrosine. In certain embodiments, conserved substitutions include amino acids that are small is size such as alanine, cysteine, glycine, proline, serine and threonine.

### Methods of Use

This disclosure relates to methods of using a guide RNA sequence and bind CRISPR associated (Cas) nucleases for the purpose of managing replication of nucleic acids or expression of genes associated therewith and thereafter optionally cleaving the nucleic acids at desired target sequences. Although it is not intended that certain embodiments of this disclosure be limited by any particular mechanism, is believed that shortening a guide sequence to partially hybridize with a target blocks cleavage of the nucleic acids and represses RNA expression.

In certain embodiments, this disclosure relates to in vitro or in vivo methods of repressing replication of a double stranded nucleic acid comprising: a) providing a cell with a double stranded nucleic acid sequence comprising a target segment adjacent to a protospacer adjacent motif (PAM) sequence or adjacent to a sequence with the reverse complement of the PAM; b) inserting into the cell an RNA sequence comprising a guide sequence under conditions such that the guide RNA sequence is expressed, wherein the guide sequence is identical to the target segment for 6 or more nucleotides but less than 17 nucleotides in length, and wherein a Cas nuclease inside the cells in combination with the guide RNA represses replication of the nucleic acid without cutting either strand of the double stranded nucleic acid.

In certain embodiments, the PAM on either a sense strand and/or a template strand can be targeted to repress transcription, so the PAM can be on either strand as long as it is opposite and adjacent to the target sequence.

In certain embodiments, the guide RNA sequence is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon. In certain embodiments, the Cas nuclease is Cas9 or Cpf1. In certain embodiments, the method further comprises inserting into the cell a Cas nuclease or a vector encoding the Cas nuclease in operable combination with a promoter.

In certain embodiments, repressing replication of a double stranded nucleic acid is repressing RNA transcription or gene expression.

In certain embodiments, inserting into the cell an RNA sequence is inserting a vector encoding the RNA sequence.

In certain embodiments, this disclosure relates to methods of cleaving a double stranded nucleic acid comprising the steps disclosed herein for blocking cleave of a target sequence further comprising inserting into the cell a second RNA sequence, or vector encoding the second RNA sequence, comprising a guide sequence under conditions such that the second RNA is expressed and wherein a Cas nuclease is inside the cell cleaves at one or both stranded of the double stranded nucleic acid.

In certain embodiments, this disclosure relates to methods of repressing replication of a double stranded nucleic acid comprising: a) providing a cell with a double stranded nucleic acid sequence comprising target segment adjacent to a protospacer adjacent motif (PAM) sequence or adjacent to a sequence with the reverse complement of the PAM; b) inserting into the cell an RNA sequence comprising a guide sequence linked to tracrRNA under conditions such that the RNA sequence is expressed in the cell, wherein the guide sequence is identical to the target sequence for 6 or more nucleotides but less than 17 nucleotides in length, wherein a Cas nuclease inside the cells, in an area of hybridization with the guide sequence, represses replication of the nucleic acid without cutting either strand of the double stranded nucleic acid.

In certain embodiments, this disclosure relates to methods of repressing replication of a double stranded nucleic acid comprising: a) providing a cell with a double stranded nucleic acid sequence comprising a target segment adjacent to a protospacer adjacent motif (PAM) sequence or adjacent to a sequence with the reverse complement of the PAM; b) inserting into the cell a vector encoding an RNA sequence comprising a guide RNA sequence under conditions such that the expression of the RNA guide is formed in the cell, wherein the guide RNA is identical to the target segment for 6 or more nucleotides but less than 17 nucleotides in length, and wherein a Cas nuclease inside the cells in combination with the guide RNA represses replication of the nucleic acid without cutting either strand of the double stranded nucleic acid.

In certain embodiments, the guide RNA is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon. In certain embodiments, the Cas nuclease is Cas9 or Cpf1. In certain embodiments, the method further comprising inserting into the cell a Cas nuclease or a vector encoding the Cas nuclease in operable combination with a promoter.

In certain embodiments, this disclosure relates to methods of cleaving a double stranded nucleic acid comprising the steps provided herein for repressing replication, further comprising inserting into the cell a second vector encoding a second RNA sequence comprising a guide RNA under conditions such that the second RNA is expressed and wherein a Cas nuclease inside the cells cleaves one or both strands of the double stranded nucleic acid.

In certain embodiments, methods of repressing replication of a double stranded nucleic acid comprises: a) providing a cell with a double stranded nucleic acid sequence comprising a target segment adjacent to a protospacer adjacent motif (PAM) sequence or adjacent to a sequence with the reverse complement of the PAM; b) inserting into the cell a vector encoding an RNA sequence comprising a guide sequence and a segment capable of hybridizing to a tracrRNA under conditions such that the expression of the RNA sequence forms a complex with a tracrRNA in the cell providing a guide RNA, wherein the guide RNA is identical to the target segment for 6 or more nucleotides but less than 17 nucleotides in length, and wherein a Cas nuclease inside the cells represses replication of the nucleic acid without cutting either strand of the double stranded nucleic acid.

In certain embodiments, the guide RNA is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon. In certain embodiments, the PAM or reverse complement of the PAM is less than 20 nucleotides upstream from a start codon.

In certain embodiments, Cas nuclease is Cas9 or Cpf1. In certain embodiments, the PAM sequence is selected from Streptococcus pyogenes, e.g., NG or NGG, NNNNGATT (SEQ ID NO: 2), Streptococcus thermophiles, e.g., NNAGAA (SEQ ID NO: 3), Treponema denticola, e.g., NAAAAC (SEQ ID NO: 4), Staphylococcus aureus, e.g., NNGRRT (R=A or G) (SEQ ID NO: 16), Campylobacter jejuni, e.g., NNNNACA (SEQ ID NO: 17), Neisseria meningitides, e.g., NNNNGATT (SEQ ID NO: 18), and Francisella orthologs, e.g., TNN or TTTN (SEQ ID NO: 19). In certain embodiments, the Cas nuclease is Cpf1 and the PAM is Streptococcus pyogenes NG or Francisella orthologs TTN.

In certain embodiments, methods disclosed herein further comprise inserting into the cell a Cas nuclease or a vector encoding the Cas nuclease in operable combination with a promoter.

In certain embodiments, methods disclosed herein further comprise inserting into the cell a guide RNA or a vector encoding the guide RNA in operable combination with a promoter.

In certain embodiments, methods disclosed herein further comprise inserting into the cell a tracrRNA or a vector encoding the tracrRNA in operable combination with a promoter.

In certain embodiments, methods disclosed herein further comprise inserting into the cell a guide RNA or a vector encoding the guide RNA in operable combination with a promoter wherein the guide RNA comprises a guide sequence linked to a tracrRNA.

In certain embodiments, methods disclosed herein further comprise inserting into the cell a guide RNA or a vector encoding the guide RNA in operable combination with a promoter wherein the guide RNA comprises a guide sequence linked to a segment of RNA that is capable of specific binding with a Cas nuclease such as a Cas9 or a Cpf1.

In certain embodiments, repressing replication of a double stranded nucleic acid is repressing RNA transcription or gene expression. In certain embodiments, the RNA is mRNA or microRNA. In certain embodiments, the double stranded nucleic acid is DNA or RNA.

In certain embodiments, a method of cleaving a double stranded nucleic acid comprises the steps disclosed herein or repressing replication of a double stranded nucleic acid, further comprising inserting into the cell a second vector encoding an RNA sequence comprising a guide RNA or guide sequence and a segment capable of hybridizing to a tracrRNA under conditions such that the guide RNA sequence is expressed and wherein a Cas nuclease inside the cells cleaves one or both strands of the double stranded nucleic acid.

In certain embodiments, a method of cleaving a nucleic a double stranded nucleic acid comprises the steps disclosed herein, further comprising inserting into the cell a second vector encoding an RNA sequence comprising a guide sequence linked to tracrRNA under conditions such that the RNA sequence is expressed in the cell and wherein a Cas nuclease inside the cells cleaves at least one stranded of the double stranded nucleic acid.

In certain embodiments, this disclosure relates to methods of cleaving a double stranded nucleic acid comprising the steps disclosed herein for repressing replication of a double stranded nucleic acid and further comprising: a) providing a cell with a double stranded nucleic acid sequence comprising target segment adjacent to a protospacer adjacent motif (PAM) sequence or adjacent to a sequence with the reverse complement of the PAM; b) inserting into the cell a vector encoding an guide RNA sequence under conditions such the RNA sequence is expressed in the cell, wherein the guide sequence is identical to the target sequence for more than 17 nucleotides in length, and wherein a Cas nuclease inside the cell cleaves at least one stranded of the double stranded nucleic acid.

In certain embodiments, the guide sequence is identical to the target segment starting in either the 5′ or 3′ direction of the PAM sequence, or starting in the 5′ or 3′ direction of the sequences with reverse complement of the PAM sequence.

In certain embodiments, the target segment does not encode a protein. In certain embodiments, the target segment is in a 5′ untranslated region of a gene upstream of a start codon.

In certain embodiments, the Cas nuclease is a Cpf1. In certain embodiments, the methods of repressing replication of a double stranded nucleic acid comprise mixing a Cas nuclease with a double stranded nucleic acid sequence comprising a sense strand target segment with a protospacer adjacent motif (PAM) sequence, wherein the PAM sequence is on the 5′ end of the target sequence, wherein mixing is with a guide RNA sequence comprising a guide sequence, wherein the guide sequence is identical to the target sequence for 6 or more nucleotides but less than 18 or 17 nucleotides in length starting in the 3′ direction of the PAM sequence, and wherein the Cas nuclease specifically binds the guide RNA repressing replication of the nucleic acid.

In certain embodiments, the Cas nuclease is a Cpf1. In certain embodiments, the methods of repressing replication of a double stranded nucleic acid comprising mixing a Cas nuclease with a double stranded nucleic acid sequence comprising a template strand target segment with a protospacer adjacent motif (PAM) sequence, wherein the PAM sequence is on the 5′ end of the target sequence, wherein mixing is with a first RNA sequence comprising a guide sequence, wherein the guide sequence is identical to the target sequence for 6 or more nucleotides but less than 18 or 17 nucleotides in length starting in the 3′ direction of the PAM sequence, and wherein the Cas nuclease represses replication of the nucleic acid.

In certain embodiments, the Cas nuclease is a Cas9. In certain embodiments, the methods of repressing replication of a double stranded nucleic acid comprising mixing a Cas nuclease with a double stranded nucleic acid sequence comprising a sense strand target segment with a protospacer adjacent motif (PAM) sequence, wherein the PAM sequence is on the 3′ end of the target sequence, wherein mixing is with a first RNA sequence comprising a guide sequence and a segment capable of hybridizing to a tracrRNA under conditions such that the RNA sequence forms a complex with a tracrRNA, wherein the guide sequence is identical to the sense strand target sequence for 6 or more nucleotides but less than 18 or 17 nucleotides in length starting in the 5′ direction of the PAM sequence, and wherein the Cas nuclease specifically represses replication of the nucleic acid.

In certain embodiments, the Cas nuclease is a Cas9. In certain embodiments, the methods of repressing replication of a double stranded nucleic acid comprise mixing a Cas nuclease with a double stranded nucleic acid sequence comprising a sense strand target segment with a protospacer adjacent motif (PAM) sequence, wherein the PAM sequence is on the 3′ end of the target sequence, wherein mixing is with a first RNA sequence comprising a guide sequence linked to a tracrRNA under conditions such that the RNA sequence forms a complex with a tracrRNA, wherein the guide sequence is identical to the sense strand target sequence for 6 or more nucleotides but less than 18 or 17 nucleotides in length starting in the 5′ direction of the PAM sequence, and wherein the Cas nuclease specifically binds the double stranded nucleic acid repressing replication of the nucleic acid.

In certain embodiments, the Cas nuclease is a Cas9. In certain embodiments, the methods of repressing replication of a double stranded nucleic acid comprise mixing a Cas nuclease with a double stranded nucleic acid sequence comprising a template strand target segment with a protospacer adjacent motif (PAM) sequence, wherein the PAM sequence is on the 3′ end of the target sequence, wherein mixing is with a first RNA sequence comprising a guide sequence and a segment capable of hybridizing to a tracrRNA under conditions such that the RNA sequence forms a complex with a tracrRNA, wherein the guide sequence is identical to the template strand target sequence for 6 or more nucleotides but less than 18 or 17 nucleotides in length starting in the 5′ direction of the PAM sequence, and wherein the Cas nuclease represses replication of the nucleic acid.

In certain embodiments, the Cas nuclease is a Cas9. In certain embodiments, the methods of repressing replication of a double stranded nucleic acid comprise mixing a Cas nuclease with a double stranded nucleic acid sequence comprising a template strand target segment with a protospacer adjacent motif (PAM) sequence, wherein the PAM sequence is on the 3′ end of the target sequence, wherein mixing is with a first RNA sequence comprising a guide sequence linked to a tracrRNA under conditions such that the RNA sequence forms a complex with a tracrRNA, wherein the guide sequence is identical to the template strand target sequence for 6 or more nucleotides but less than 18 or 17 nucleotides in length starting in the 5′ direction of the PAM sequence, and wherein the Cas nuclease specifically binds the double stranded nucleic acid repressing replication of the nucleic acid.

In certain embodiments, the sense strand or template strand target sequence does not encode a protein. In certain embodiments, the target sequence contains the start codon of a protein. In certain embodiments, the target sequence is an untranslated region up-stream from a start codon, such as in a promotor region. In certain embodiments, the target sequence comprises sequences of cis-acting regulatory elements such as promotor, enhancer, and silencer sequences. In certain embodiments, the target sequence comprises a ribosome binding site (RBS) sequence, Shine Dalgarno sequence AGGAGGU (SEQ ID NO: 11), Kozak consensus sequence ACCAUGG (SEQ ID NO: 12), or contains the start codon. In certain embodiments, the sense strand target sequence comprises a TATA (SEQ ID NO: 1) box, TATAWAW (SEQ ID NO: 13), where W is either A or T, Pribnow box, TATAAT (SEQ ID NO: 14), CCAAT (SEQ ID NO: 15) box.

With regard to any of the embodiments disclosed herein, the target segment is in a 5′ untranslated region of a gene upstream of a start codon or transcription start site such as a promotor region sequence. In certain embodiments, the target segment has a PAM or reverse complement of the PAM is less than 20 nucleotides upstream from a start codon. In certain embodiments, the target segment is less than 19 nucleotides upstream from a start codon. In certain embodiments, the target segment is less than 18 nucleotides upstream from a start codon. In certain embodiments, the target segment is less than 17 nucleotides upstream from a start codon. In certain embodiments, the target segment is less than 16 nucleotides upstream from a start codon. In certain embodiments, the target segment is less than 15 nucleotides upstream from a start codon. In certain embodiments, the target segment is less than 14 nucleotides upstream from a start codon. In certain embodiments, the target segment is less than 13 nucleotides upstream from a start codon. In certain embodiments, the target segment is less than 12 or 11 nucleotides upstream from a start codon.

In certain embodiments, the target segment has a PAM or reverse complement of the PAM in a 5′ untranslated region and the PAM is less than 20 nucleotides upstream from the start codon. In certain embodiments, the target segment is in a 5′ untranslated region and the PAM or reverse complement is less than 30 nucleotides upstream from the start codon. In certain embodiments, the target segment is in a 5′ untranslated region and the PAM or reverse complement is less than 40 nucleotides upstream from the start codon. In certain embodiments, the target segment is in a 5′ untranslated region and the PAM or reverse complement is less than 50 nucleotides upstream from the start codon. In certain embodiments, the target segment is in a 5′ untranslated region and the PAM or reverse complement is less than 100 nucleotides upstream from the start codon. In certain embodiments, the 5′ untranslated region and the PAM or reverse complement is less than 200 nucleotides upstream from the start codon. In certain embodiments, the 5′ untranslated region and the PAM or reverse complement is less than 300 nucleotides upstream from the start codon. In certain embodiments, the 5′ untranslated region and the PAM or reverse complement is less than 400 nucleotides upstream from the start codon. In certain embodiments, the 5′ untranslated region and the PAM or reverse complement is less than 500 nucleotides upstream from the start codon. In certain embodiments, the 5′ untranslated region and the PAM or reverse complement is less than 1000 nucleotides upstream from the start codon. In certain embodiments, the 5′ untranslated region and the PAM or reverse complement is less than 1500 nucleotides upstream from the start codon. In certain embodiments, the 5′ untranslated region and the PAM or reverse complement is less than 2500 nucleotides upstream from the start codon.

In certain embodiments, this disclosure relates to in vitro or in vivo methods of repressing replication of a double stranded nucleic acid comprising mixing a Cas nuclease, which is a Cpf1, with a double stranded nucleic acid sequence comprising a target segment with a protospacer adjacent motif (PAM) sequence, wherein the PAM sequence is on the 5′ end of the target sequence, wherein mixing is with an first RNA sequence comprising a guide sequence, wherein the guide sequence is identical to the sense strand target sequence for 6 or more nucleotides but less than 18 or 17 nucleotides in length starting in the 3′ direction of the PAM sequence, and wherein the Cas nuclease specifically binds the double stranded nucleic acid repressing replication of the nucleic acid without cleaving the nucleic acid. In certain embodiments, the target sequence does not encode a protein. In certain embodiments, the target sequence is an untranslated region upstream from a start codon, such as in a promotor region.

In certain embodiments, this disclosure relates to in vitro or in vivo methods of repressing replication of a double stranded nucleic acid comprising mixing a Cas nuclease, which is a Cas9, with a double stranded nucleic acid sequence comprising a target segment with a protospacer adjacent motif (PAM) sequence, wherein the PAM sequence is on the 3′ end of the target sequence, wherein mixing is with an first RNA sequence comprising a guide sequence linked to tracrRNA, wherein the guide sequence is identical to the sense strand target sequence for 6 or more nucleotides but less than 18 or 17 nucleotides in length starting in the 5′ direction of the PAM sequence, and wherein the Cas nuclease specifically binds the double stranded nucleic acid repressing replication of the nucleic acid without cleaving the nucleic acid. In certain embodiments, the target sequence does not encode a protein. In certain embodiments, the target sequence is an untranslated region up-stream from a start codon, such as in a promotor region.

In certain embodiments, this disclosure relates to methods of cleaving a double stranded nucleic acid comprising the steps of repressing replication of a double stranded nucleic acid as disclosed herein, further comprising mixing a second RNA sequence comprising a guide sequence and a segment capable of hybridizing to a tracrRNA under conditions such the expression of the RNA sequence forms a complex with a tracrRNA, wherein the guide sequence is identical to the target sequence for 18 or 17 or more nucleotides and wherein a Cas nuclease cleaves at least one stranded of the double stranded nucleic acid. In certain embodiments, the method comprises cleaving both strands of the nucleic acid. In certain embodiments, the method comprises cleaving the sense strand of the nucleic acid. In certain embodiments, the method comprises cleaving the template strand of the nucleic acid.

In certain embodiments, the target sequence is in the sense strand or template strand, the Cas nuclease is Cas9 and target sequence for 18 or 17 or more nucleotides in length starting in the 5′ direction of the PAM sequence.

In certain embodiments, the target sequence is in the sense strand or template strand, the Cas nuclease is Cpf1 and target sequence for 18 or 17 or more nucleotides in length starting in the 3′ direction of the PAM sequence.

In certain embodiments, this disclosure relates to methods of repressing replication of a double stranded nucleic acid comprising: a) providing a cell with a double stranded nucleic acid sequence comprising a target segment with a protospacer adjacent motif (PAM) sequence, wherein the PAM sequence is on the 3′ end or 5′ end of the target sequence; b) inserting into the cell a vector encoding an RNA sequence comprising a guide sequence linked to tracrRNA under conditions such the RNA sequence is expressed in the cell, wherein the guide sequence is identical to the sense strand target sequence for 6 or more nucleotides but less than 18 or 17 nucleotides in length starting in the 3′ or 5′ direction of the PAM sequence, wherein a Cas nuclease inside the cells specifically binds the double stranded nucleic acid repressing replication of the nucleic acid without cleaving the nucleic acid. In certain embodiments, the target sequence does not encode a protein. In certain embodiments, the target sequence is an untranslated region up-stream from a start codon, such as in a promotor region.

In certain embodiments, this disclosure relates to methods of cleaving a double stranded nucleic acid comprising the steps of repressing replication of a double stranded nucleic acid disclosed herein, further comprising inserting into the cell a second vector encoding an RNA sequence comprising a guide sequence and a segment capable of hybridizing to a tracrRNA under conditions such the expression of the RNA sequence forms a complex with a tracrRNA in the cell, wherein the guide sequence is identical to the target sequence for 17 or more nucleotides in length starting in the 5′ or 3′ direction of the PAM sequence or reverse complement, and wherein a Cas nuclease inside the cells cleaves at least one stranded of the double stranded nucleic acid. In certain embodiments, the method comprises cleaving both strands of the nucleic acid. In certain embodiments, the method comprises cleaving the sense strand of the nucleic acid. In certain embodiments, the method comprises cleaving the template strand of the nucleic acid.

In certain embodiments, this disclosure relates to methods of cleaving a nucleic a double stranded nucleic acid comprising the steps of repressing replication of a double stranded nucleic acid disclosed herein, further comprising inserting into the cell a second vector encoding an guide RNA sequence or and RNA sequence comprising a guide sequence linked to tracrRNA under conditions such the RNA sequence is expressed in the cell, wherein the guide sequence is identical to the target sequence for 18 or 17 or more nucleotides in length starting in the 5′ or 3′ direction of the PAM sequence or reverse complement, and wherein a Cas nuclease inside the cells cleaves one or both strands of the double stranded nucleic acid. In certain embodiments, the method comprises cleaving the sense strand of the nucleic acid. In certain embodiments, the method comprises cleaving the template strand of the nucleic acid.

In certain embodiments, this disclosure relates to methods of repressing replication of a double stranded nucleic acid without cleaving the nucleic acid comprising: a) providing a cell with a double stranded nucleic acid sequence comprising a target segment with a protospacer adjacent motif (PAM) sequence, wherein the PAM sequence is on the 3′ end or 5′ end of the target sequence; b) inserting into the cell a vector encoding an RNA sequence comprising a guide RNA or guide sequence and a segment capable of hybridizing to a tracrRNA under conditions such the expression of the RNA sequence forms a complex with a tracrRNA in the cell, wherein the guide sequence is identical to the target sequence for 6 or more nucleotides but less than 18 or 17 nucleotides in length starting in the 5′ direction or 3′ direction of the PAM sequence or reverse complement, and wherein a Cas nuclease inside the cells represses replication of the nucleic acid. In certain embodiments, the target sequence does not encode a protein. In certain embodiments, the target sequence is an untranslated region up-stream from a start codon, such as in a promotor region.

In certain embodiments, this disclosure relates to methods of repressing replication of a double stranded nucleic acid comprising: a) providing a cell with a double stranded nucleic acid sequence comprising a protospacer adjacent motif (PAM) sequence or reverse complement adjacent to a target sequence, wherein the PAM sequence or reverse complement is on the 3′ end or 5′ end of a target sequence; b) inserting into the cell a single stranded RNA sequence optionally linked to tracrRNA and/or inserting into the cells a vector encoding a Cas nuclease, e.g., Cas9 or Cpf1, and optionally a single stranded RNA sequence comprising a guide sequence optionally linked to tracrRNA, wherein the guide sequence is identical to the target sequence for 6 or more nucleotides but less than 18 or 17 nucleotides in length adjacent to and starting in the 5′ direction or 3′ direction of the PAM sequence or reverse complement, wherein the Cas nuclease specifically binds the double stranded nucleic acid without cleaving the double stranded nucleic acid repressing replication of the nucleic acid. In certain embodiments, repressing replication of a double stranded nucleic acid is repressing RNA transcription, expression or gene expression. In certain embodiments, the RNA is mRNA or microRNA. In certain embodiments, the sense strand target sequence does not encode a protein. In certain embodiments, the sense strand target sequence is an untranslated region up-stream from a start codon, such as in a promotor region.

In certain embodiments, this disclosure relates to methods of repressing replication of a double stranded nucleic acid comprising: a) providing a cell with a double stranded nucleic acid sequence comprising a target segment with a protospacer adjacent motif (PAM) sequence, wherein the PAM sequence is on the 3′ end or 5′ end of the target sequence; b) inserting into the cell a single stranded RNA sequence optionally linked to tracrRNA or a vector encoding a single stranded RNA sequence comprising a guide sequence optionally linked to tracrRNA, wherein the guide sequence is identical to the target sequence for 6 or more nucleotides but less than 18 or 17 nucleotides in length adjacent to and starting in the 5′ direction or 3′ direction of the PAM sequence or reverse complement, wherein the single stranded RNA, double stranded nucleic acid, and a tracrRNA forms a complex that specifically binds a Cas nuclease without cleaving the double stranded nucleic acid repressing replication of the nucleic acid. In certain embodiments, repressing replication of a double stranded nucleic acid is repressing RNA expression or gene expression. In certain embodiments, the sense strand target sequence does not encode a protein. In certain embodiments, the sense strand target sequence is an untranslated region upstream from a start codon, such as in a promotor region.

In certain embodiments, the double stranded nucleic acid is DNA or RNA. In certain embodiments, the double stranded nucleic acid is human, viral or bacterial, DNA or RNA. In certain embodiments, repressing replication is preventing or slowing a segment of DNA or RNA from being copied into RNA by an RNA polymerase. In certain embodiments, the RNA is mRNA or microRNA.

In certain embodiments, the protospacer adjacent motif (PAM) sequence is any PAM sequence associated with a Cas9 or Cpf1.

In certain embodiments, the guide sequence for repressing replication is only identical to the target coding sequence for between 7 and 16 nucleotides, or 7 and 15 nucleotides, or 8 and 16 nucleotides, or 8 and 15 nucleotides in length starting in the 5′ direction or 3′ direction of the PAM sequence or reverse complement of the PAM.

In certain embodiments, the method further comprises inserting into the cell a Cas nuclease, such as Cas9 or Cpf1, or a vector encoding the Cas nuclease, such as Cas9 or Cpf1, in operable combination with a promoter. In certain embodiments, the method further comprises inserting into the cell tracrRNA or a vector encoding tracrRNA in operable combination with a promoter. In certain embodiments, the method further comprises inserting into the cell sgRNA (guide sequence linked to a tracrRNA) or a vector encoding sgRNA in operable combination with a promoter.

In certain embodiments, this disclosure relates to methods of repressing gene expression, mRNA or microRNA expression of a gene comprising: a) providing a cell with a double stranded nucleic acid sequence encoding a gene comprising a target segment with a protospacer adjacent motif (PAM) sequence or reverse complement, wherein the PAM sequence is on the 3′ end or 5′ end of the target sequence; b) inserting into the cell a single stranded gRNA sequence optionally linked to tracrRNA or a vector encoding a single stranded RNA sequence comprising a guide RNA sequence optionally linked to tracrRNA, wherein the guide RNA sequence is only identical to the target sequence for 5 or 6 or more nucleotides but less than 18 or 17 nucleotides in length starting in the 5′ direction or 3′ direction of the PAM sequence or reverse complement, wherein the single stranded RNA, double stranded nucleic acid, and a tracrRNA forms a complex that specifically binds a Cas nuclease, such as Cas9 or Cpf1, without cleaving the double stranded nucleic acid repressing replication of the nucleic acid. In certain embodiments, the sense strand target sequence does not encode a protein. In certain embodiments, the sense strand target sequence is an untranslated region up-stream from a start codon, such as in a promotor region.

In certain embodiments, the method further comprises inserting into the cell a Cas nuclease, such as Cas9 or Cpf1, or a vector encoding the Cas nuclease, such as Cas9 or Cpf1, in operable combination with a promoter. In certain embodiments, the method further comprises inserting into the cell tracrRNA or a vector encoding tracrRNA in operable combination with a promoter.

In certain embodiments, this disclosure relates vectors encoding a Cas nuclease and RNA reported herein. In certain embodiments, the vectors contain heterologous nucleic acid sequences such that the entire vector is not naturally occurring.

In certain embodiments, the method further comprises inserting into the cell a Cas nuclease, such as Cas9, or a vector encoding the Cas nuclease, such as Cas9, in operable combination with a promoter. In certain embodiments, the method further comprises inserting into the cell tracrRNA or a vector encoding tracrRNA in operable combination with a promoter.

In some aspects, the disclosure provides methods comprising delivering one or more nucleic acids, such as or one or more vectors as described herein, one or more transcripts thereof, and/or one or proteins transcribed therefrom, to a cell. In certain embodiments, a single vector encodes a Cas nuclease and a guide RNA as described herein optional in operable combination with the same or different promoters. In certain embodiments, a first vector encodes a Cas nuclease, and a second vector encodes a guide RNA as described herein e.g., the guide RNA and tracrRNA as linked or separate, optional in operable combination with the same or different promoters. In certain embodiments, a first vector encodes a Cas nuclease, and a second vector encodes a guide RNA and a third vector encodes a tracrRNA as described herein.

In some aspects, the disclosure further provides cells produced by such methods, and organisms (such as animals, plants, or fungi) comprising or produced from such cells. In some embodiments, a Cas nuclease in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™)

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity tier up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian immunodeficiency virus (SIV), human immunodeficiency virus (HIV), and combinations thereof. In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids. e.g., in the in vitro production of nucleic acids and peptides, and for in vivo and ex vivo gene

Packaging cells are typically used to form virus particles that are capable of infecting a host cell. Such cells include 293 cells, which package adenovirus, and PA317 which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21. BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In certain embodiments, this disclosure contemplates using methods disclosed herein for transcriptional control using a guide RNA with a shorter target sequence in combination with a catalytically active Cas nuclease for improved activation and repression. For CRISPR-based repression, Cas nucleases are able to efficiently block transcriptional elongation or initiation in prokaryotes. However, as evidenced with dCas9 and other nuclease-inactive systems, the level of repression is reduced in eukaryotic cells. To overcome this limitation, Cas nucleases can be modified to improve interaction with eukaryotic promoter and transcription initiation regions of the DNA. These modifications include fusing the catalytically active Cas nuclease to a Kruppel-associated box (KRAB), Max-interacting protein 1 (Mxi1), or four concatenated mSin3 interaction domains (SID4X). Transcriptional activation can be achieved by using the nuclease to prevent binding of a transcriptional repressor, or using the Cas nuclease to recruit transcriptional activators. Cas nucleases can be fused to various proteins to improve the efficiency of activation in different cell types and to recruit additional cellular proteins to specific sites on the DNA. In certain embodiments, this disclosure contemplates using same Cas nuclease to both cleave DNA and control transcription, guided by different RNAs.

In certain embodiments, this disclosure contemplates using methods disclosed herein for multiplexing. In certain embodiments, this disclosure contemplates expressing a Cas nuclease with multiple different guide RNAs so that it can be multiplexed for different targets. In certain embodiments, this disclosure contemplates using catalytically active Cas nuclease with different types of guide RNAs, the protein could be multiplexed for many different functions that that could be executed simultaneously by a single type of Cas nuclease include spacers designed to cleave, repress, activate, detect, and degrade single or double stranded nucleic acids, e.g. dsDNA and ssDNA.

In certain embodiments, this disclosure contemplates using multiple CRISPR proteins at the same time: multiple nucleases could be used simultaneously in the cell to further fine tune the nucleic acids in the cell. For example, Cas9 and Cpf1 could be delivered to the cell, each with multiple RNA guides or using fusion guide RNAs.

In certain embodiments, this disclosure contemplates Cpf1 processes its own target-specific crRNAs from the CRISPR array. This activity is independent of the nuclease domains. Cpf1 can be provided with a single CRISPR array containing spacers designed to cleave, repress, activate, detect, and degrade ssDNA. Because the DNA and RNA cleaving enzymatic domains are intact, Cpf1 can process its own crRNAs from a multiplexed array and execute diverse cellular functions based on the characteristics of the guide RNA and targeted sequence.

In certain embodiments, this disclosure contemplates providing a single CRISPR-Cas nuclease array with multiple spacers would be an alternative to providing a Cas nuclease, e.g., Cpf1 or Cas9, with multiple separately transcribed crRNAs.

### Methods to Regulate Viral Transcription

In certain embodiments, this disclosure relates to compositions for treating a viral infection. Compositions include a vector comprising nucleic acids that encodes a Cas nuclease and a guide sequence to targeting sequence in a viral genome wherein the guide sequence is identical to the target sequence for 6 or more nucleotides but less than 17 nucleotides in length starting in the 5′ or 3′ direction of the PAM sequence or reverse complement, wherein the Cas nuclease binds to the target in the viral genome via the targeting sequence and affects transcription of at least a portion of the viral genome. In some embodiments, the complex inhibits transcription of at least a portion of the viral genome. A targeting sequence may be used that matches the target according to predetermined criteria and does not match any portion of a host genome.

In preferred embodiments, the virus is capable of latent infection of a human host. Suitable targets include: a preC promoter in a hepatitis B virus (HBV) genome; an 51 promoter in the HBV genome; an S2 promoter in the HBV genome; and an X promoter in the HBV genome; a viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; and an HPV late promoter.

The composition may be provided within a carrier such that it is suitable for topical application to the human skin. In some embodiments, the nucleic acid is within a plasmid that is carried and delivered to the human skin by the carrier.

Any suitable virus can be targeted such as, for example, adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapneumovirus, Hendra virus, nipah virus, rabies virus, hepatitis D virus, rotavirus, orbivirus, coltivirus, or banna virus.

In some embodiments, this disclosure provides for methods of treating a viral infection. The method includes introducing into a host cell or administering to a subject in need thereof an effective amount of a composition comprising nucleic acid that encodes a Cas nuclease, and a guide RNA or sgRNA that targets a viral genome. The Cas nuclease binds to the RNA to form a complex, and the complex hybridizes to the target in the viral genome via a targeting sequence within the RNA. The complex inhibits transcription of at least a portion of the viral genome. Preferably, viral infection is a latent or active infection. Introducing the composition into the host cell may include delivering the composition to a local reservoir of latent infection within a human patient. The target in the viral genome may include any of a preC promoter in a hepatitis B virus (HBV) genome; an S1 promoter in the HBV genome; an S2 promoter in the HBV genome; and an X promoter in the HBV genome; the viral Cp (C promoter) in an Epstein-Barr virus genome; a minor transcript promoter region in a Kaposi's sarcoma-associated herpesvirus (KSHV) genome; a major transcript promoter in the KSHV genome; an Egr-1 promoter from a herpes-simplex virus (HSV); an ICP 4 promoter from HSV-1; an ICP 10 promoter from HSV-2; a cytomegalovirus (CMV) early enhancer element; a cytomegalovirus immediate-early promoter; an HPV early promoter; or an HPV late promoter.

In some embodiments, the method includes using the complex to cause upregulation of transcription within the host cell. For example, the Cas nuclease/gRNA complex may bind copies of the nucleic acid that encodes either or both of those components and upregulate their own further expression. Thus, in some embodiments, wherein a double stranded nucleic acid is part of a plasmid, wherein the Cas nuclease binds to guide RNA to form a complex, the complex hybridizes to the plasmid causing up-regulated transcription of at least a portion of the plasmid. In certain embodiments, an initial transcription of the plasmid within the host cell results in a positive feedback cycle in which the up-regulated transcription then increases the up-regulated transcription. In certain embodiments, the transcription can be terminated by adding a guide RNA causing cleave of the plasmid causing up-regulated transcription.

### Multi-Capable Cas Nucleases for DNA Cleavage and Transcriptional Control

CRISPR systems prevents DNA cleavage of the CRISPR array in their own genomes by a matching crRNA:Cas9 complex. Self-cleavage is avoided because Cas9 uses two stages of target recognition. Before the spacer on the crRNA can interact with the complementary sequence on the target DNA (called the protospacer), Cas9 first has to recognize a short three nucleotide sequence on the opposite strand of the target DNA from where the crRNA binds. For Cas9, this three-nucleotide sequence is nGG. The Cas9 protein hops along the DNA until it sees an nGG sequence, and when it does, it changes conformation slightly to interact more strongly with the nGG and separate the two DNA strands. Then, if the crRNA bound to Cas9 has a spacer that matches the target DNA directly adjacent to the PAM, the crRNA binds and Cas9 is conformationally activated to cleave the DNA.

A natural system in has been identified which Cas9 can regulate RNA expression by binding to the DNA directly downstream of the promoter and preventing transcriptional read-through. However, it does so using a catalytically active Cas9, which is unique from CRISPRi technologies. Francisella Cas9 uses two different RNA complexes, one with tracrRNA and crRNA to cleave foreign DNA, and another with tracrRNA and scaRNA (another small RNA transcribed near the CRISPR array) that interacts with the promoter DNA but does not cleave it, to prevent transcription. Disclosed herein is the engineering of a multi-functional Cas9, a Cas9 protein that is capable of both transcriptional control by biding to the DNA and foreign DNA cleavage, and these functions can be defined by the RNAs bound to Cas9.

The way that Cas9 is able to bind DNA but not cleave in order to repress RNA transcription is by using a spacer sequence with partial complementarity to its target DNA sequence. Because the spacer does not contain the full 18-20 base pairs of complementarity Cas9 needs to induce a double strand break in the target sequence, Cas9 is able to bind to the target DNA without cleaving. However, when Cas9 is supplied with a spacer sequence that fully matches its target, it is able to cleave. Cas9 can therefore be used to explore more complex aspects of physiology that benefit from simultaneous RNA and DNA regulation. The use of a dual functional Cas9 for engineering is unexplored and has enormous applications. These uses include but are not limited to improved antivirals and antimicrobials, optimization in the food and fermentation industries, anti-cancer therapeutics, and a tool for studying basic biological questions and for making mutations in genes that first require repression of another gene or system. By activating or inactivating using a ligand, or even using inducible promoters to alter which RNA is expressed, the function of Cas9 in a cell could be modulated temporally and spatially. Until now, in order to repress transcription and cleave a DNA target by Cas9, two different variants of the Cas9 protein would need to be expressed within the cell. Cas9 is a very large protein, so doubling the concentration in the cell increases the toxicity and complications associated with delivery, expression, and maintenance of Cas9 within the cell. Now, it is possible to have both functions from a single protein by simply altering the associated RNA sequence.

A Cas9 protein that is capable of DNA target cleavage is also able to bind to DNA to repress RNA transcription without an alteration to the protein. To do this, one modifies the length of the target sequence encoded on the scaRNA or other guide RNA. Other work on using Cas9 for transcriptional activation or repression use a modified Cas9 that is unable to cleave DNA, called the dCas9. dCas9 has mutations in the RuvC and HNH active sites, allowing the protein to bind but not cleave DNA. Transcriptional activation and repression by a dCas9 is often referred to as CRISPRa or CRISPRi.

CRISPR systems must prevent DNA cleavage of the CRISPR array in their own genomes, as the crRNA sequences bound to Cas9 are identical to both the foreign nucleic acid target and the CRISPR array in the bacterial chromosome. Cas9 uses two stages of target recognition to avoid self-cleavage. Before the spacer on the crRNA can interact with the complementary sequence on the target DNA (called the protospacer), Cas9 first has to recognize a short three nucleotide sequence on the opposite strand of the target DNA. For Cas9, this sequence is -nGG. Only when the crRNA bound to Cas9 has a spacer that matches the target DNA is directly adjacent to a PAM on the opposite strand, can Cas9 cleave the DNA.

The CRISPR-Cas9 system from Francisella novicida is especially interesting because it plays an important role in the pathogenic lifecycle of the bacteria in addition to foreign DNA cleavage. F. novicida Cas9 (FnoCas9) has a secondary function regulating an endogenous mRNA encoding a bacterial lipoprotein (BLP), 1103. 1103 BLP regulation by FnoCas9 is modulated by tracrRNA and scaRNA, which is an additional small RNA transcribed from the CRISPR locus. Together, FnoCas9 and these RNAs enable robust repression of 1103 transcript levels. Francisella novicida is a facultative intracellular pathogen that must passage through the macrophage phagosome without activating the innate immune system in order to cause an infection in a mammalian host. Cas9 mutants of F. novicida are highly attenuated in a mouse model of infection, which is in part mediated by the hyper-activation of the innate immune response caused by 1103 overexpression.

Experimental data indicates that 1103 repression is mediated by a mechanism of Cas9 tracrRNA:scaRNA interaction with the mRNA, however the precise molecular mechanism of this regulation has remained elusive. Interestingly, RNA targeting mechanisms have been identified for other Cas9 orthologs.

SpyCas9 can target RNA when supplied with a short PAMmer sequence, while NmeCas9, SauCas9 and CjeCas9 have recently been shown degrade ssRNA in a PAM-independent manner that requires perfect or near perfect interaction with the crRNA spacer and the HNH catalytic domain. ssRNA cleavage has been proposed to have implications in endogenous gene regulation (CjeCas9) and foreign nucleic acid defense (SauCas9). However, FnoCas9 is not capable of this mechanism of ssRNA targeting.

FnoCas9 can inhibit HCV using an engineered guide RNA (rgRNA) with a 20 bp spacer for a viral UTR target. Notably, HCV is an RNA virus and thus lacks a DNA stage, suggesting that an FnoCas9:RNA complex is capable of interaction with RNA. FnoCas9 was also effective in inhibiting RNA viral infections of tobacco mosaic virus and cucumber mosaic virus in plants. However, the magnitude of viral control in these engineered systems was 2-5 fold, while Cas9 regulation of 1103 is 2.5-3 log. RNA requirements for RNA transcript repression in F. novicida was incomplete. Thus, other regulatory targets of FnoCas9 were studied to help elucidate the precise molecular mechanism of endogenous gene regulation by FnoCas9.

The specificity of endogenous gene regulation by FnoCas9 was identified by mapping the transcriptome. FnoCas9 maintains an exquisitely specific regulon and employs a unique mechanism of endogenous of gene regulation that is independent of ssRNA cleavage. Interestingly, the mechanism of FnoCas9 endogenous gene regulation is PAM-dependent and uses a catalytically active Cas9 that is capable of RNA repression and DNA cleavage.

Using an analysis of the FnoCas9 native transcriptome to identify the specificity of endogenous gene regulation, one is able to locate the region required for transcriptional repression of two distinct transcripts to the 5′ UTR of each mRNA. FnCas9 uses a secondary RNA, scaRNA, to interact with the template strand of the 5′ UTR by initial recognition of a PAM followed by recognition of the target DNA with a partial spacer sequence located on the tail of scaRNA. By interacting with the 5′ UTR, Cas9 represses transcriptional read-though. FnoCas9 does this to regulate endogenous genes with remarkably high specificity.

FnoCas9 mediates the transcriptional repression using a cleavage capable Cas9. By modifying the targeting sequence of the RNA guide, FnoCas9 is able to alternate function between repression of transcription and DNA cleavage, without detrimental effects for the cell. The ability of a shorter RNA to guide Cas9 to bind but not cleave a DNA target has been shown in vitro, and is believed to be the result of Cas9 being unable to enter into a fully cleavage-capable state. Similarly, modulation of the length of scaRNA-target complementarity can be used to modulate the level of mRNA expression in F. novicida. The partial sequence similarity prevents lethal cleavage of the chromosome while also stably regulating RNA levels for long-term F. novicida physiology.

Interestingly, this system is unique from engineered CRISPRi systems, which use a dCas9 (catalytically inactive protein) in complex with an RNA guide with 20 bp of complementarity for its target. Like in these studies, dCas9 does not alter repression 1103 by Cas9. Interestingly, in these systems the guide RNA length is not altered to modulate repression level, rather it is distance from the TSS that is modulated. The sensitivity of natural FnoCas9 regulation was in the length of the RNA-target interaction, but that generally, proximity of the TSS is important for repression to occur, but not for altering the sensitivity of repression.

Using an analysis of the native FnoCas9 transcriptome to elucidate the specificity of endogenous gene regulation, the site of interaction between the FnoCas9 complex and the DNA of the 5′ UTR of each transcript was located in its regulon. FnoCas9 uses scaRNA to interact with the template strand of the 5′ UTR by recognition of a PAM and a scaRNA-complementary sequence on the DNA target. By targeting the 5′ UTR DNA, FnoCas9 functions as a transcription factor, repressing gene expression. Through this interaction, FnoCas9 regulates the expression of four endogenous genes with remarkably high specificity. Repression is dependent on a PAM in the 5′ UTR and that the sensitivity of natural FnoCas9 regulation could be modulated by the length of the RNA-target interaction and proximity of the scaRNA binding site to the TSS. Further, transcriptional repression by FnoCas9 could be achieved through the targeting of either strand. The extent of complementarity between scaRNA and the DNA target alters the binding affinity of the dual-RNAFnoCas9 complex to the DNA. The distance of the TSS from the scaRNA target region does not affect the binding affinity of the complex to the DNA. Using this knowledge of scaRNA:tracrRNA-FnoCas9 interaction with DNA, scaRNA was reprogrammed such that FnoCas9 targeted the promoters of desired genes to repress transcription, highlighting the potential use of scaRNA:tracrRNA-FnoCas9 in the control of gene expression.

It is particularly interesting that in spite of the degeneration of its repeat sequence, scaRNA has retained the ability to direct DNA cleavage. When F. novicida is transformed with an artificial target containing 20 bases of identity to the scaRNA, FnoCas9 restricts transformation. However, the 11 consecutive bases of perfect complementarity between scaRNA and the native 1104 and 1101 5′ UTRs is sufficient for robust transcriptional repression, which may be due to the inability of FnoCas9 to enter a cleavage-favorable conformation with a partial scaRNA-DNA target interaction. Thus, modification of the length of the targeting sequence of the guiding scaRNA:tracrRNA duplex determines whether FnoCas9 represses transcription or cleaves its DNA target. The scaRNA was reprogrammed to target genes involved in polymyxin resistance. This led to efficient repression of the targeted genes and greatly increased sensitivity to polymyxin.

F. novicida utilizes two distinct RNA duplexes for foreign DNA restriction and transcriptional repression, although both are capable of DNA restriction. The prevalence of these systems and the minimal base-pair requirements needed for a shift between DNA cleavage and transcriptional interference suggest that a role of Cas9 as a transcriptional regulator may be a broader phenomenon in bacterial physiology than previously expected.

### Examples

Genomic Locus Encoding Genes that are Repressed by Cas9, tracrRNA and scaRNA

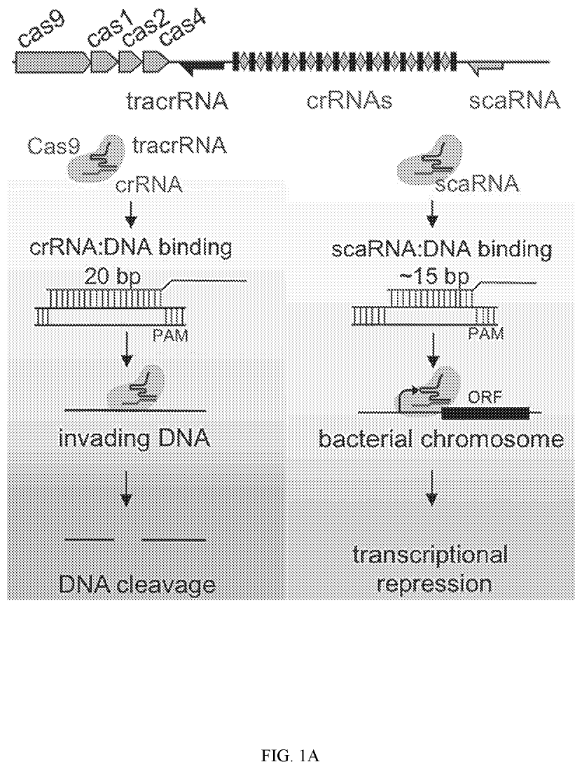

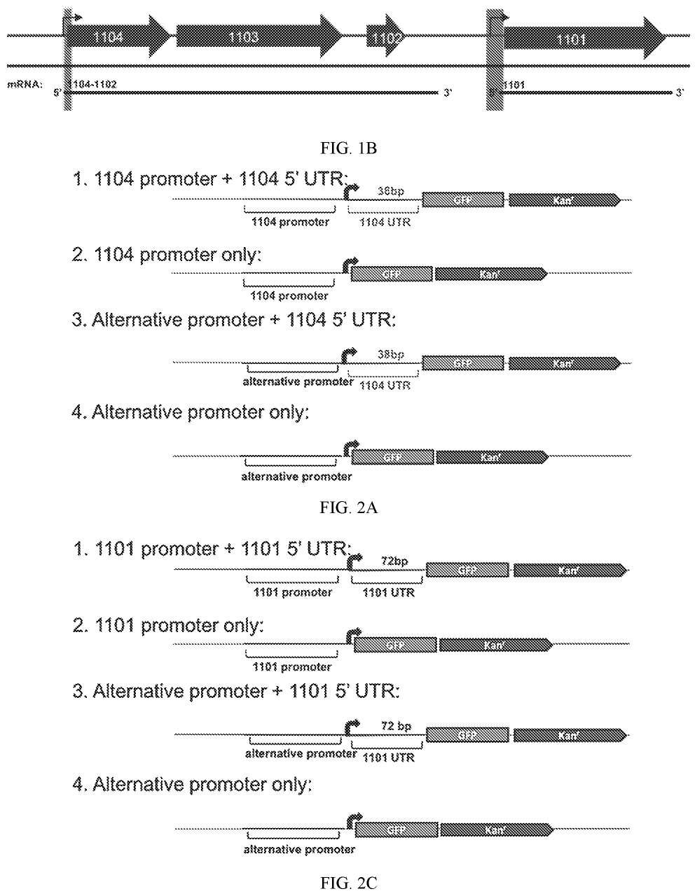

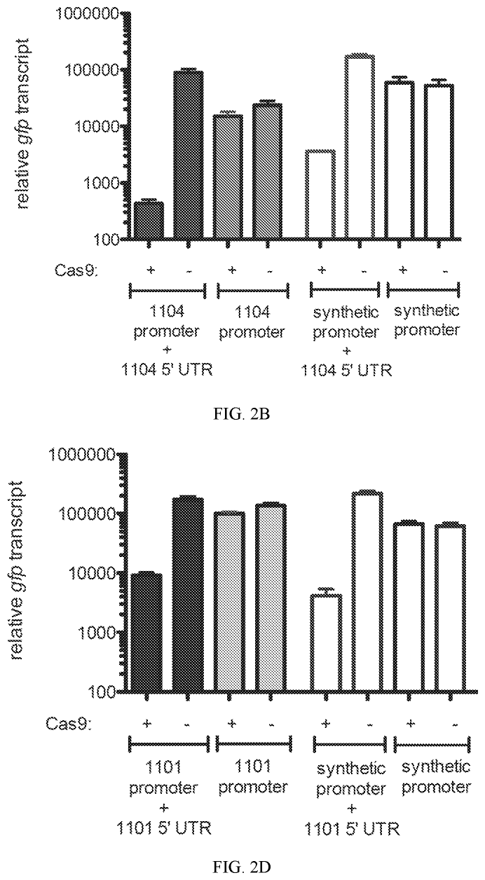

Cas9 and two RNAs, tracrRNA and scaRNA, were identified as required for the repression of 4 Francisella novicida genes, named 1101-1104. These genes are encoded on two RNA transcripts by PCR, called 1101 and 1104-1102 (FIG. 1). From systematic mutations of the coding and non-coding regions of this DNA locus, it was identified that the 5′ untranslated region (UTR) was involved in recognition of these sequences by Cas9 (depicted in FIG. 1). To identify whether the 38 base pair (bp) UTR of 1104 and the 72 bp UTR in the 1101 mRNA could confer Cas9-dependent repression of a non-native sequence from a non-native promoter, a series of promoter fusion constructs were made in the genome of F. novicida (FIG. 2A and FIG. 2B). It was discovered the UTR by itself was enough to enable Cas9 to repress the levels of an RNA transcript (FIG. 2C and FIG. 2D).

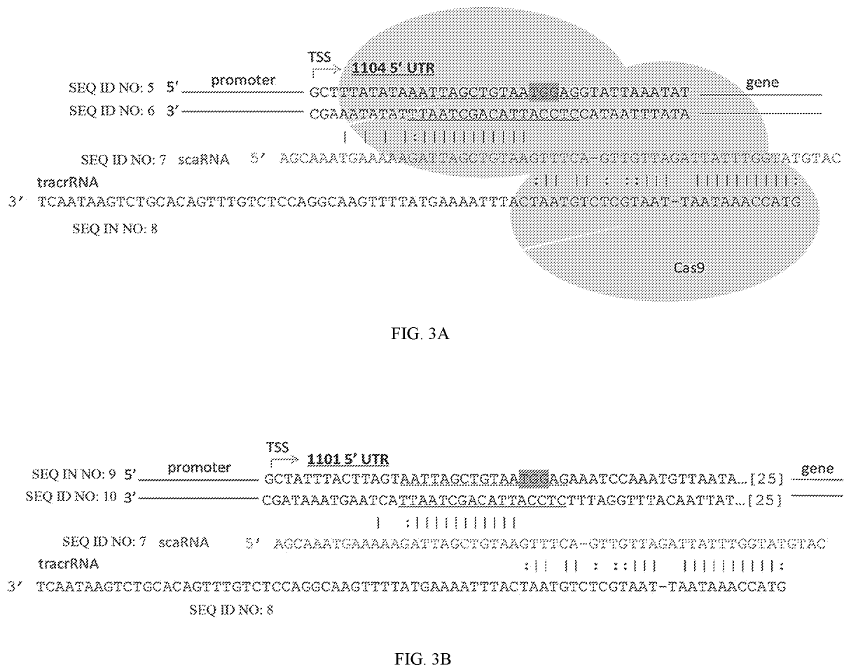

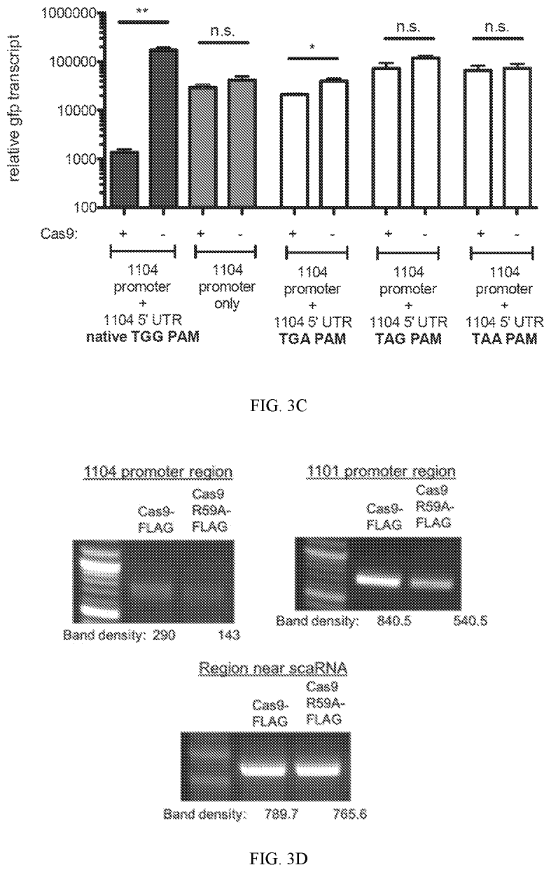

The tracrRNA and scaRNA were mapped to the UTR sequences to look for a binding interaction. It was discovered that the scaRNA interacts with the template strand of the DNA of the 1101 and 1104 UTR (FIG. 3A and FIG. 3B). Mutation of the PAM that is located on the opposite strand of the DNA from where scaRNA binds is enough to fully prevent Cas9 from inhibiting transcription (FIG. 3C). Cas9 is not targeting the RNA in this case because it is binding to the template strand of the DNA, which is not the strand encoded in the mRNA. It was determined that Cas9 interacts with DNA at the UTR region by comparing the ability of a wild-type Cas9 protein and a Cas9 mutant (called Cas9:R59A) to bind DNA in the UTR. The studies indicate that the inability of Cas9 to bind to tracrRNA and scaRNA reduced its ability to interact with the DNA of the 1101 and 1104 UTR, specifically (FIG. 3D). Thus, one can use a partial binding sequence on an sgRNA or RNA duplex such as tracrRNA: scaRNA to interact with a target DNA downstream of the transcriptional start site to prevent transcription read-through.

### FnoCas9 Transcriptional Interference is Controlled by Target Proximity to the TSS

The importance of proximity of the 5′ UTR to the transcriptional start site (TSS) was tested by measuring gfp* transcript level from fusion constructs with either 0, 5, 10, or 20 bases between the TSS of the synthetic constitutive promoter (p146) and the 1104 5′ UTR region with complementarity to scaRNA. Constructs with 0, 5, and 10 bases between the TSS and the scaRNA complementarity region effectively repressed gfp*. However, the construct with 20 bp between the TSS and the scaRNA complementarity region exhibited significantly reduced repression. These data highlight that the scaRNA complementarity region must be in close proximity to the TSS for effective FnoCas9-dependent transcriptional interference to occur.

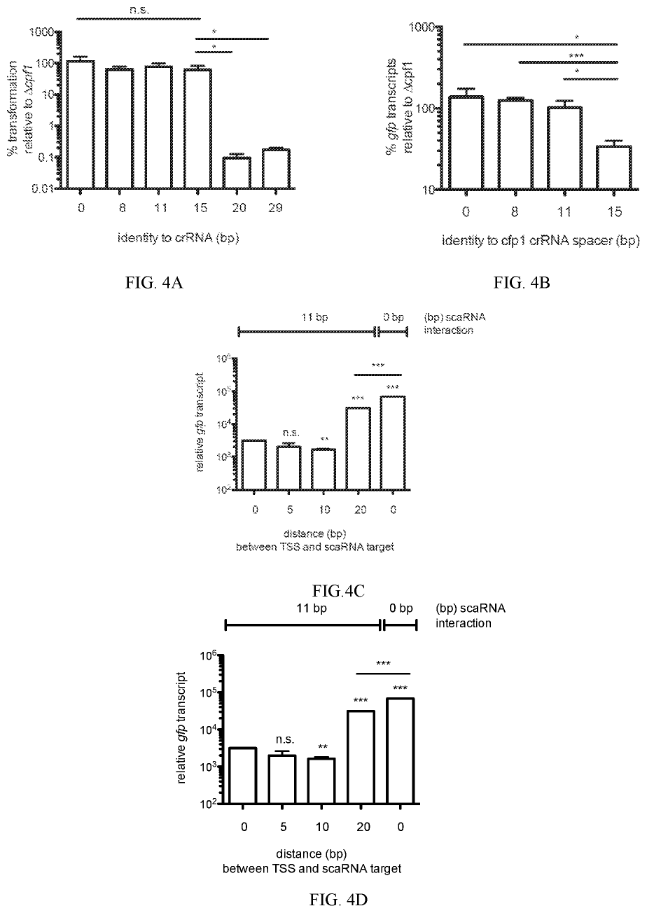

The importance of proximity of the 5′ UTR to the transcriptional start site (TSS) was tested by measuring gfp* transcript level from fusion constructs with either 0, 5, 10, or 20 bases between the TSS of the synthetic constitutive promoter (p146) and the 1104 5′ UTR region with complementarity to scaRNA. Constructs with 0, 5, and 10 bases between the TSS and the scaRNA complementarity region effectively repressed gfp* (FIG. 4A). However, the construct with 20 bp between the TSS and the scaRNA complementarity region exhibited significantly reduced repression (FIG. 5). These data highlight that the scaRNA complementarity region must be in close proximity to the TSS for effective FnoCas9-dependent transcriptional interference to occur.

scaRNA can be Reprogrammed to Guide Cas9 to Repress Other Virulence Factors

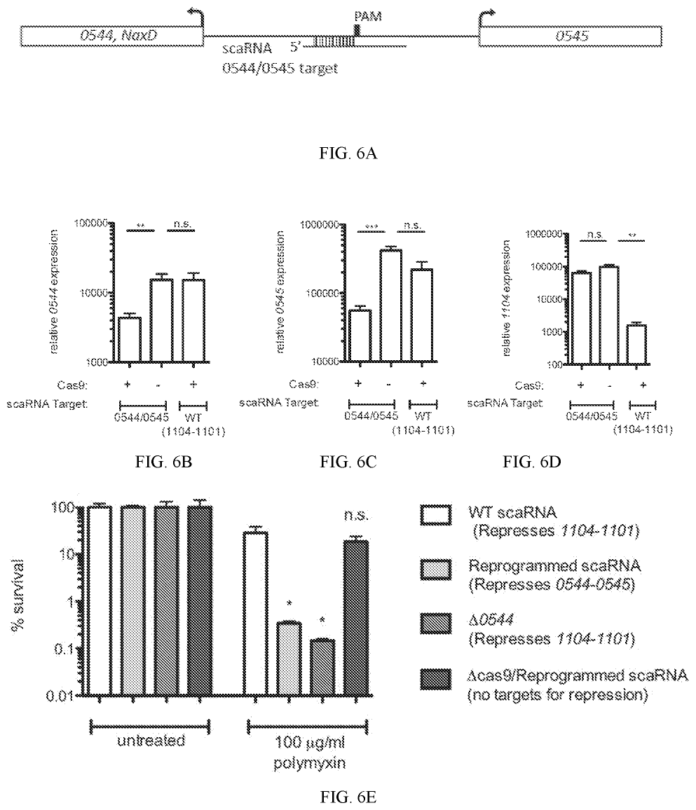

To determine if scaRNA could be reprogrammed to repress new targets, the 1104-1101 targeting scaRNA tail was replaced with a 15 bp sequence to target the intergenic region between FTN_0544 and 0545. The sequence adjacent to the most central PAM to the two genes was targeted. These two genes are transcribed in opposite directions, with a 98 base pair stretch between the two ORFs (FIG. 6A). These two genes are required for the modification of outer membrane lipid A that leads to resistance to the antibiotic polymyxin B; they are transcribed in opposite directions and are not regulated by FnoCas9. Mutation of either genes results in attenuation of F. novicida in a mouse model, and generally alters envelope physiology. Interestingly, in a strain with scaRNA reprogrammed for 0544 and 0545, a significant reduction in transcript levels was observed compared to WT and a Cas9 mutant (FIG. 6B-C). When scaRNA was reprogrammed for 0544/0545, 1104 was de-repressed due to the new target site for scaRNA (FIG. 6D). These results indicate that Cas9 can be reprogrammed to robustly repress expression from new targets, and when targeted to the same site in the intergenic region between two nearby genes. The repression of 0544 and 0545 in the scaRNA reprogrammed strain led to an increased susceptibility to polymyxin B of almost 100-fold, similar to a 0544 deletion strain (FIG. 6E). The susceptibility was reversed by deletion of cas9 from the reprogrammed scaRNA strain (FIG. 6E). These results indicate that FnoCas9 can be reprogrammed to repress expression from new targets in a scaRNA:tracrRNA-dependent manner, highlighting the scaRNA:tracrRNA-FnoCas9 machinery as a potential new tool to control gene expression and modulate bacterial physiology.

Cpf1 Represses Transcription from a Plasmid Using a Partial crRNA Sequence that is Unable to Inhibit Transformation with the Plasmid.

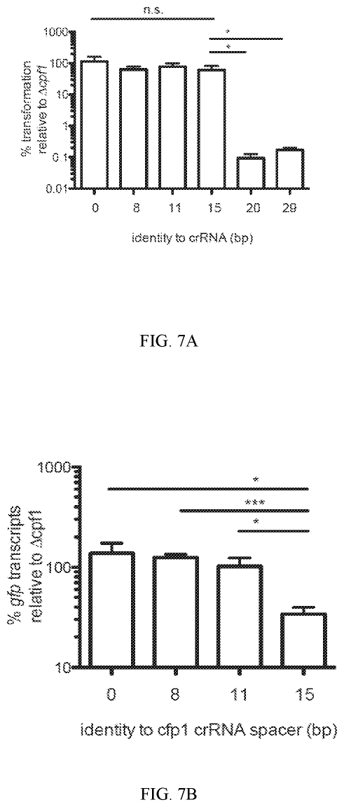

Plasmid constructs were designed with gfp under the control of a synthetic constitutive promoter for Francisella novicida. Between the transcriptional start site of the promoter and gfp, we placed the F. novicida Cpf1 PAM (FnoCpf1, protospacer adjacent motif) followed by 0, 8, 11, 15, 20 or 29 bases of complementarity to a native FnoCpf1 crRNA spacer. 29 bp is the full length of the native spacer. These constructs were transformed into wild-type (WT) and a cpf1 mutant of F. novicida and the ability of WT to restrict transformation with each construct was enumerated in FIG. 7A as % transformants recovered from WT relative to a cpf1 mutant. Constructs with 0, 8, 11, and 15 bp of crRNA spacer identity transformed with the same efficiency into both WT and a cpf1 mutant. However, Cpf1 was able to restrict transformation into WT when the plasmid contained 20 and 29 bases of identity to crRNA. We then cultured successful transformants from WT and cfp1 containing either the 0, 8, 11, and 15 bp plasmids, and measured the level of gfp mRNA by qRT-PCR. The results are enumerated in FIG. 7B as % gfp transcripts recovered from WT relative to a cpf1 mutant. gfp expression from the plasmid with 15 bases of complementarity to a Cpf1 spacer is reduced, suggesting that the partial crRNA is guiding Cpf1 to bind but not to cleave at the promoter of the plasmid.

1. A method of modulating replication of a double stranded nucleic acid comprising:
a) providing a cell with a double stranded nucleic acid sequence comprising a target segment adjacent to a protospacer adjacent motif (PAM) sequence or adjacent to a sequence with the reverse complement of the PAM;
b) inserting into the cell a vector encoding an RNA sequence comprising a guide sequence under conditions such that the RNA sequence is expressed;
wherein the guide sequence is identical to the target segment for 6 or more nucleotides but less than 17 nucleotides in length, and wherein a Cas nuclease inside the cells in combination with the guide sequence represses replication of the nucleic acid without cutting either strand of the double stranded nucleic acid.

2. The method of claim 1 wherein the guide sequence is identical to the target segment starting in either the 5′ or 3′ direction of the PAM sequence, or starting in the 5′ or 3′ direction of the sequences with reverse complement of the PAM sequence.

3. The method of claim 1, wherein the target segment does not encode a protein.

4. The method of claim 1, wherein the target segment is in a 5′ untranslated region of a gene upstream of a start codon.

5. The method of claim 1, wherein the PAM or reverse complement of the PAM is less than 20 nucleotides upstream from a start codon.

6. The method of claim 1, wherein the Cas nuclease is Cas9 or Cpf1.

7. The method of claim 1 further comprising inserting into the cell a Cas nuclease or a vector encoding the Cas nuclease in operable combination with a promoter.

8. The method of claim 1, wherein repressing replication of a double stranded nucleic acid is repressing RNA transcription or gene expression.

9. The method of claim 1, wherein the 6 or more nucleotides but less than 17 nucleotides in length are contiguous.

10. A method of cleaving a nucleic a double stranded nucleic acid comprising the steps provided in claim 1, further comprising inserting into the cell a second vector encoding a second RNA sequence comprising a guide sequence under conditions such that the second RNA is expressed and wherein a Cas nuclease inside the cells cleaves at least one stranded of the double stranded nucleic acid.

11. A method of modulating replication of a double stranded nucleic acid comprising:
a) providing a cell with a double stranded nucleic acid sequence comprising target segment adjacent to a protospacer adjacent motif (PAM) sequence or adjacent to a sequence with the reverse complement of the PAM;
b) inserting into the cell a vector encoding an RNA sequence comprising a guide sequence linked to tracrRNA under conditions such the RNA sequence is expressed in the cell,
wherein the guide sequence is identical to the target sequence for 6 or more nucleotides but less than 17 nucleotides in length, wherein a Cas nuclease inside the cells represses replication of the nucleic acid without cutting either strand of the double stranded nucleic acid.

12. The method of claim 11 wherein the guide sequence is identical to the target segment starting in either the 5′ or 3′ direction of the PAM sequence, or starting in the 5′ or 3′ direction of the sequences with reverse complement of the PAM sequence.

13. The method of claim 11, wherein the target segment does not encode a protein.

14. The method of claim 11, wherein the target segment is in a 5′ untranslated region of a gene upstream of a start codon.

15. The method of claim 11, wherein the PAM or reverse complement of the PAM is less than 20 nucleotides upstream from a start codon.

16. The method of claim 11, wherein the Cas nuclease is Cas9.

17. The method of claim 11, further comprising inserting into the cell a Cas nuclease or a vector encoding the Cas nuclease in operable combination with a promoter.

18. A method of cleaving a nucleic a double stranded nucleic acid comprising the steps provided in claim 11, further comprising inserting into the cell a second vector encoding a second RNA sequence comprising a guide sequence linked to tracrRNA under conditions such that the second RNA is expressed and wherein a Cas nuclease inside the cells cleaves at least one stranded of the double stranded nucleic acid.

19. A method of repressing replication of a double stranded nucleic acid comprising:
a) providing a cell with a double stranded nucleic acid sequence comprising target segment adjacent to a protospacer adjacent motif (PAM) sequence or adjacent to a sequence with the reverse complement of the PAM;
b) inserting into the cell a vector encoding an RNA sequence comprising a guide sequence and tracrRNA under conditions such the guide sequence and tracrRNA is expressed in the cell,
wherein the guide sequence is identical to the target sequence for 6 or more nucleotides but less than 17 nucleotides in length, wherein a Cas nuclease inside the cells represses replication of the nucleic acid without cutting either strand of the double stranded nucleic acid.

20. A method of cleaving a nucleic a double stranded nucleic acid comprising the steps provided in claim 19, further comprising inserting into the cell a second vector encoding a second RNA sequence comprising a guide sequence under conditions such that the second RNA is expressed and wherein a Cas nuclease inside the cells cleaves at least one stranded of the double stranded nucleic acid.
